(19) United States
(12) Patent Application Publication
(10) Pub. No.: US 2008/0045485 A1
(43) Pub. Date: Feb. 21, 2008

(54) Methods of diagnosing synovial disease in a mammal by detecting bacterial DNA in synovial tissues from dogs with inflammatory knee arthritis and degenerative anterior cruciate ligament rupture

(76) Inventor: Peter Muir, Madison, WI (US)

Correspondence Address: QUARLES & BRADY LLP, 33 E. MAIN ST, SUITE 900, P.O. BOX 2113, MADISON, WI, 53701-2113, US

(21) Appl. No.: 11/893,740

(22) Filed: Aug. 17, 2007

Related U.S. Application Data
(60) Provisional application No. 60/838,469, filed on Aug. 17, 2006.

(51) Int. Cl. A61K 31/65; A61P 19/02; C12Q 1/02; C12Q 1/42; C12Q 1/68; G01N 33/48; G01N 33/68

(52) U.S. Cl. 514/152; 435/21; 435/29; 435/6; 436/86; 436/94

(57) ABSTRACT

Method of diagnosing persistent, chronic synovitis and progressive joint degradation in the joint of a mammal. The method including providing a test sample comprising synovial fluid, cell or tissue from the joint of a mammal; detecting the presence of bacterial DNA, measuring the concentration of one or more biomarkers being cathepsin K, MMP-2 and -9, cathepsin S, tartrate-resistant acid phosphatase, invariant chain, CD4⁺ T-lymphocytes, CD8⁺ T-lymphocytes, CD44⁺ mononuclear cells, Toll-like receptor-2, Toll-like receptor-9, or a combination thereof; and, comparing the concentration of the biomarker from the test sample to a corresponding biomarker concentration in a control sample from healthy dogs, or an internal PBMC control sample, wherein a statistically significant elevated concentration of the biomarker in the test sample indicates that the mammal's joint is diseased.

### CROSS-REFERENCE TO RELATED APPLICATIONS

This application claims priority to U.S. Provisional Patent Application Ser. No. 60/838,469 filed on Aug. 17, 2006.

U.S. Patent Application Publication No. US 2005/0074800 A1 was published on Apr. 7, 2005, which is incorporated herein by reference.

### FIELD OF THE INVENTION

The field of the invention relates to kits and methods for detecting synovial disease in the knee of a human or the stifle dog. The anatomically correct term is “knee” for humans and “stifle” for dogs.

### BACKGROUND OF THE INVENTION

It has been reported that in the US more than $1 billion is spent annually treating ruptured ligaments in dogs. It has been generally assumed that such ruptures are due to trauma or natural tissue degeneration. However, increasing evidence suggests that there may be an immunological component to such ligament disease. That link is based on findings showing large amounts of macrophages, dendritic cells, and T lymphocytes in the synovial tissues of affected joints.

Rupture of the cranial cruciate ligament (CCL) is the most significant orthopedic disease in pet dogs. Although the cause of this disease is unknown, it is generally accepted that rupture of the ligament is associated with the development of an inflammatory arthritis and the presence of large numbers of macrophages, dendritic cells, and T lymphocytes in the synovium of the stifle. Taken together, these data suggest that there is an immune-mediated mechanism to this disease. Any immunologic trigger for this disease is not known.

There exists a need to understand how polymicrobial populations of bacteria interact with the immune system to promote synovitis in mammals. Arthritis affects about 1 in 3 adult Americans and is the leading cause of disability. Host-pathogen interactions are a key factor in the development of arthritis. Multiple genera of bacteria may be associated with the development of synovial inflammation, particularly in the knee joint. Development of joint inflammation may also depend on host genetics. However, any specific mechanism involving bacterial triggering of joint inflammation is not well understood.

Immune system dysregulation may be a key component concerning the pathogenesis of rheumatoid arthritis (“RA”); the most important chronic inflammatory arthritis condition in human beings. Reactive arthritis may sometimes follow bacterial infection, but the triggering agent is generally unknown.

Lyme disease often leads to the development of inflammatory arthritis whereby the triggering agent is known to be Borrelia burgdorferi species complex. Lyme disease is important because it is a very common vector-borne disease, particularly in the U.S. Borrelia spirochaetes are transmitted to the mammalian host via a bite from an Ixodes tick. Arthritis is also a common consequence of Borrelia infection which can be recurrent in nature. In a proportion of patients infected with Lyme disease, persistent and severe synovitis develops that is refractory to known treatments. Although not widely studied, it is likely that Borrelia burgdorferi infection occurs as part of a polymicrobial population of joint bacteria, which likely acts to promote dysregulation of joint immune responses to bacterial infection.

Regardless of how synovitis is triggered, the synovial pathology is similar in all forms of chronic inflammatory arthritis. The synovium shows hypertrophy, vascular proliferation and the presence of many dendritic cells. The synovium also shows synovial macrophages and T and B lymphocytes suggesting an antigen-driven process that can induce dysregulation of the immune system.

In recent years, considerable progress has been made in showing that bacteria are pivotal to the development of chronic inflammatory arthritis. In cases of Lyme arthritis and reactive arthritis, synovial inflammation recedes eventually disappearing within months to several years suggesting that bacterial killing ultimately limits the duration of joint inflammation.

In contrast, joint inflammation in RA is more persistent and may become life-long suggesting ongoing immune stimulation perhaps from an unidentified infectious agent. Interaction between bacteria and the host immune system is complex. For example, it is known that humans having specific major histocompatibility complex (“MHC”) class II alleles are predisposed to immune-mediated joint disease. However, it is currently unclear what specific immune mechanism perpetuates joint inflammation in all forms of chronic inflammatory arthritis.

Current hypotheses for such specific immune mechanism include: Infection-induced autoimmunity; antigen-specific T lymphocyte epitope mimicry in humans having specific MHC class II alleles, particularly those containing the RA shared epitope; bystander activation of innate or adaptive immune responses by bacteria in an antigen-nonspecific mechanism; and persistent immune stimulation from sequestered bacterial antigens. There is an important and long-felt need to better understand the immune mechanism so as to prevent development and progression of various forms of treatment-resistant chronic inflammatory arthritis and associated disabilities.

In 1973, Pond and Nuki made a seminal observation that blind acute transection of the canine anterior cruciate ligament (ACL) led to knee instability and rapid development of osteoarthritis. On the basis of that study, it was concluded that the canine model was well-suited to studies of the pathogenesis and treatment of osteoarthritis. Since then, the canine model has been widely used in orthopaedic research.

In the 1970's and 1980's, it was also recognized that naturally occurring rupture of the ACL was an important cause of lameness in domestic dogs. By extrapolation from clinical thinking about ACL rupture in humans, it has been generally accepted that rupture of the ACL had a similar etiology in both humans and dogs and that the rupture mechanism was primarily traumatic rather than degenerative.

In 1985, it was further recognized that use of the Pond-Nuki model in dogs induced obvious snyovial inflammation in the stifle. (Lipowitz et al., Synovial membrane changes after experimental transaction of the cranial cruciate ligament in dogs, Am J Vet Res 1985, 46:1166-1170). However, synovitis in the contralateral stifle remains unexplained. (Myers et al., Synovitis and osteoarthritic changes in canine articular cartilage after anterior cruciate ligament transection, Arthritis Rheum 1990, 33:1406-15). Patella tendon and fascia lata autografts have been widely used for surgically treating domestic dogs having naturally occurring ACL ruptures. Critical evaluation of those results may not be available in abundance. (Conzemius et al., Effect of surgical technique on limb function after surgery for rupture of the cranial cruciate ligament in dogs, J Am Vet Med Assoc 2005, 226:232-236).

Further examination of dogs having naturally occurring ACL rupture has recognized a few common features: Rupture of the ACL during normal activity; bilateral knee arthritis; bilateral ACL rupture; and, moderate to severe arthritis at the time of clinical diagnosis of the ACL rupture. In 1988, these common features were reappraised, and it was hypothesized that naturally occurring ACL rupture in dogs developed because of progressive degeneration of the ACL over time rather than due to accidental injury. (Bennett et al., A reappraisal of anterior cruciate ligament disease in the dog, J Small Anim Pract 1988, 29:275-297). Others found that material properties of the canine ACL declined with aging in association with development of chondroid transformation of ligament fibroblasts, loss of fibroblasts, and disruption of collagen fibers within the ACL. (Vasseur et al., Correlative biomechanical and histologic study of the cranial cruciate ligament in dogs, Am J Vet Res 1985, 46:1842-1854).

During the early 1990's, veterinary orthopaedic surgeons also discovered that in cases where second-look arthrotomies were performed on canine patients having patellar tendon or fascia lata autographs, graft material had often resorbed. That result was in contrast to other reported results. (Arnocsky et al., Anterior cruciate ligament replacement using patellar tendon, J Bone Joint Surg 1982, 64A:217-224). Chronic recurrent inflammation and joint effusion following ACL reconstruction is considered common in humans by some orthopaedic surgeons. (Kaab et al., Coincidence of recurrent arthritis and Behcet's disease following anterior cruciate ligament reconstruction, Arthroscopy 2002, 18:E1-5). However, most laboratories studying human ACL rupture and ligament reconstruction are studying ACL histology and biomechanics. (Allen et al., Injury and reconstruction of the anterior cruciate ligament and knee osteoarthritis, Osteoarthritis Cartilage 1999, 7:110-121).

The potential role of knee joint synovial inflammation in the ACL rupture mechanism and the development of graft laxity and failure after reconstructive surgery have not received significant recognition. From the mid-1990's to present, some in the field of canine ACL biology have recognized that naturally occurring ACL rupture is associated with chronic inflammation of the synovium, accumulation of macrophages, B and T lymphocytes, dendritic cells, and immunoglobulins. (Lawrence et al., Elevation of immunoglobulin deposition in the synovial membrane of dogs with cranial cruciate ligament rupture, Vet Immunol Immunopathol 1998, 65:89-96; and, Lemburg et al., Immunohistochemical characterization of inflammatory cell populations and adhesion molecule expression in synovial membranes from dogs with spontaneous cranial cruciate ligament rupture, Vet Immunol Immunopathol 2004, 97:231-240).

Other studies have suggested that tartrate-resistant acid phosphatase (TRAP⁺) and cathepsin K⁺ mononuclear cells accumulate in the synovium and ACL epiligament in dogs having ACL rupture. (Muir et al., Evaluation of tartrate-resistant acid phosphatase and cathepsin K in ruptured cranial cruciate ligament in dogs, Am J Vet Res 2002, 63:1279-1284; and, Muir et al., Localization of cathepsin K and tartrate-resistant acid phosphatase in synovial membrane and cranial cruciate ligament in dogs with cruciate disease, Vet Surg 2005, 34:239-246). Development of ACL hypoxia and cell necrosis may mediate loss of ligament fibroblasts from the ACL and chondroid transformation of surviving cells. (Hayashi et al., Histologic changes in ruptured canine cranial cruciate ligament, Vet Surg 2003, 32:269-277; and, Hayashi et al., Ligament fibroblast viability in ruptured canine cranial cruciate ligament, Am J Vet Res 2003, 64:1010-1016).

TRAP⁺ mononuclear cells are derived from recruitment and activation of peripheral blood mononuclear cells (PBMC) within the synovium. TRAP⁺ mononuclear cells are considered key features of destructive inflammatory arthritis. (Tsuboi et al., Tartrate-resistant acid phosphatase (TRAP) positive cells in rheumatoid synovium may induce the destruction of articular cartilage, Ann Rheum Dis 2003, 62:196-203). Although the biological function of TRAP in activated macrophages is not well understood, TRAP may have a specific functional role in antigen-specific immune responses to bacteria. (Hayman et al., Osteoclastic tartrate-resistant acid phosphatase (Acp 5): its localization in dendritic cells and diverse murine tissues, J Histochem Cytochem 2000, 48:219-227; and, Bune et al., Mice lacking tartrate-resistant acid phosphatase (Acp 5) have disordered macrophage inflammatory responses and reduced clearance of the pathogen, Staphylococus aureus, Immunology 2001, 102:103-113). Such findings suggest that pathogenesis of inflammatory arthritis associated with naturally occurring degenerative ACL rupture in dogs is immune-mediated.

Pre-existing joint inflammation may precipitate progressive degradation of the ACL eventually inducing ACL rupture during normal activity due to progressive cleavage of collagen within the ACL. Progressive collagen degradation secondary to joint inflammation will induce degraded mechanical properties of the ligament. (Goldberg et al., The influence of experimental immune synovitis on failure model and strength of the rabbit anterior cruciate ligament, J Bone Joint Surg 1982, 64A:900-906).

Few studies have attempted to determine the antigenic trigger mechanism concerning canine ACL rupture. Quantification of antibodies to type I and type II collagen in canine stifles with and without ACL rupture (and other canine joints with osteoarthritis) has demonstrated increased collagen autoantibodies in arthritic joints as compared to normal joints. (de Rooster et al., Prevalence and relevance of antibodies to type-I and type-II collagen in synovial fluid of dogs with cranial cruciate ligament damage, Am J Vet Res 2000, 61:1456-1461). Such quantification suggests that exposure of collagen neoepitopes occur secondary to joint degradation and also suggesting that it probably is not a trigger antigen of interest.

The role of bacteria in the pathogenesis of immune-mediated inflammatory arthritis is actively studied. Previous studies suggest that immune dysregulation is an important factor mediating progressive destruction of the stifle joint in dogs having degenerative naturally occurring ACL rupture. However, the precise immune mechanism by which such dysregulation occurs is unknown. Several investigators have used PCR methods to detect bacterial DNA implicating the presence of bacteria as an important causative factor concerning joint inflammation. (Gerard et al., Chromosomal DNA from a variety of bacterial species is present in synovial tissue from patients with various forms of arthritis, Arthritis Rheum 2001, 44:1689-1697).

Bacterial products within a synovial joint (such as lipoproteins and DNA) are major virulence factors that may induce joint injury and/or provoke persistent joint inflammation. Lipoproteins and DNA may bind directly to toll-like receptors on dendritic cells, macrophages, and regulatory T lymphocytes via antigen-nonspecific mechanisms. (Deng et al., Synovial cytokine mRNA expression during arthritis triggered by CpG motifs of bacterial DNA, Arthritis Res 2001, 3:48-53; Vasselon et al., Toll receptors: a central element in innate immune responses, Infect Immun 2002, 70:1033-1041; Batsford et al., Outer surface lipoproteins of Borrelia burgdorferi vary in their ability to induce experimental joint injury, Arthritis Rheum 2004, 50:2360-2369; Zanin-Zhorov et al., Heat shock protein 60 enhances CD4+CD25+ regulatory T cell function via innate TLR-2 signaling, J Clin Invest 2006, 116:2022-2032).

Activation of synovial macrophages can be mediated by bacterial infection. Arthritogenic and intracellular bacteria may also induce expression of TRAP in monocyte-macrophages. (Weiss et al., Sequential patterns of gene expression by bovine monocyte-derived macrophages associated with ingestion of mycobacterial organisms, Microb Pathog 2004, 37:215-224; and, Zhang et al., Synovial fibroblasts infected with Salmonella enterica serovar typhimurium mediate osteoclast differentiation and activation, Infect Immun 2004, 72:7183-7189).

Through an epitope mimicry scenario, specific bacterial products may provoke subsequent autoimmune inflammation. That hypothesis is controversial in the area of rheumatology. However, some evidence suggests that such processes are key concerning chronic Lyme arthritis in humans through mimicry between an immunodominant T cell epitope of outer surface lipoprotein A (Osp A) and human lymphocyte function-associated antigen 1. (Gross et al., Identification of LFA-1 as a candidate autoantigen in treatment-resistant Lyme arthritis, Science 1998, 281:703-706; Steere et al., Autoimmune mechanisms in antibiotic treatment-resistant lyme arthritis, J Autoimmun 2001, 16:263-268; Benoist et al., Autoimmunity provoked by infection: how good is the case for T cell epitope mimicry?, Nature Immunol 2001, 2:797-801; and, Gomes et al., IFN_{γ} production in peripheral blood of early Lyme disease patients to hLFAα_{L} (aa326-345), BMC Musculoskelet Disord 2002, 3:25-37).

Other research has identified that treatment-resistant Lyme arthritis develops preferentially in human patients having specific MHC class II polymorphisms, particularly human leucocyte antigen HLA-DRB1*04 and *01. (Kalish et al., Association of treatment-resistant chronic Lyme arthritis with HLA-DR4 and antibody reactivity to OspA and OspB of Borrelia burgdorferi, Infect Immun 1993, 61:2774-2779; and, Steere et al., Association of chronic, treatment-resistant Lyme arthritis with rheumatoid arthritis alleles [abstract], Arthritis Rheum 1998, 41:S81).

Such research has been key to the autoimmune hypotheses particularly because these same alleles (which are part of the RA shared epitope) may also promote joint destruction in patients having RA. (Wagner et al., Prospective analysis of the impact of HLA-DR and -DQ on joint destruction in recent-onset rheumatoid arthritis, Rheumatology 2003, 42:553-562). Apparently, such investigations have not been performed in dogs. Susceptibility to RA in humans and susceptibility to inflammatory small joint polyarthritis in dogs may be associated with a conserved amino acid motif (QRRAA and RKRAA) in the third hypervariable region of a number of HLA-DRB1 and dog leucocyte antigen DLA-DRB1 alleles. (Gregersen et al., The shared epitope hypothesis: an approach to understanding the molecular genetics of susceptibility to rheumatoid arthritis, Arthritis Rheum 1987, 30:1205-1213; Ollier et al., Dog MHC alleles containing the human RA shared epitope confer susceptibility to canine rheumatoid arthritis, Immunogenetics 2001, 53:669-673; and, Tezenas du Montcel et al., New classification of HLA-DRB1 alleles supports the shared epitope hypothesis of rheumatoid arthritis susceptibility, Arthritis Rheum 2005, 52:1063-1068).

That region of the gene may encode a functionally important part of the DRB1 molecule influencing peptide binding and interfacing between the T-cell receptor and the MHC/peptide. (Barber et al., Contribution of T-cell receptor-containing and peptide-binding residues of the class II molecule HLA-DR4Dw10 to serologic and antigen-specific T-cell recognition, Hum Immunol 1991, 32:110-118).

The relevance of canine MHC Class II genotype to the canine inflammatory stifle arthritis/ACL rupture model has also been a focus of research. Bacterial material (such as lipoproteins and/or DNA) may directly stimulate the immune system and provoke local joint injury. However, there remains an important gap in knowledge concerning work performed in rodent models of inflammatory arthritis and persistent synovial inflammation found in human patients having various forms of chronic knee arthritis, chronic Lyme arthritis, reactive arthritis, undifferentiated oligoarthritis, and RA.

There exists a long felt need for an improved animal model to study interactions between bacterial triggers and subjects having well-defined MHC class II alleles, sequence homology equivalent to human genes, and a high incidence of homozygosity. (Kennedy et al., Extensive interbreed, but minimal intrabreed, variation in DLA class II alleles and haplotypes in dogs, Tissue Antigens 2002, 59:194-204). The MHC complex may be a highly conserved group of genes having a critical role concerning adaptive immunity. (Glimcher et al., Sequences and factors: A guide to MHC class II transcription, Annu Rev Immunol 1992, 10:13-49; Meyer et al., How selection shapes variation of the human major histocompatibility complex: a review, Ann Hum Genet 2001, 65:1-26; Kennedy et al., Extensive interbreed, but minimal intrabreed, variation in DLA class II alleles and haplotypes in dogs, Tissue Antigens 2002, 59:194-204; Ting et al., Genetic control of MHC class II expression, Cell 2002, 109:S21 -S33; and, Wagner J L, Molecular organization of the canine major histocompatability complex, J Hered 2003, 94:23-26).

MHC class II genes encode cell surface molecules that are constitutively expressed in dogs in antigen-presenting cells, such as dendritic cells, macrophages and B and T lymphocytes. That presentation of self and non-self antigens to the immune system may be critical to the regulation of immune responses. Expression may also be induced in other MHC class II negative cells where different cells exhibit different patterns of responses to inflammatory cytokines like interferon γ.

MHC class II molecules bind and present foreign peptide antigens to CD4⁺ T lymphocytes. Extensive variation has been found in the peptide binding regions at several MHC loci in humans and dogs. MHC class II alleles protect against infection, whereby heterozygosity is advantageous. Precise regulation of MHC class II expression may be required for normal immune system function. Dysregulation may lead to severe immunodeficiency or autoimmune disease. Expression of MHC class II molecules is carefully controlled at the transcriptional level. The MHC class II transactivator (CIITA) is a most important protein that interacts with promotors of class II genes.

Studies of RA-associated HLA-DRB1 molecules suggest that multiple mechanisms may influence disease at all levels from peptide binding, selection of CD4⁺ T cells, and peripheral activation of T-cell dependent responses. (Buckner et al., Genetics of rheumatoid arthritis: Is there a scientific explanation for the human leukocyte antigen association? Curr Opin Rheumatol 2002, 14:254-259). Whether bacterial antigens are a unique/critical or key arthritogenic peptide or nucleic acid trigger for RA remains unclear and controversial.

Hence, there exists a long felt need to understand inflammatory stifle arthritis and degenerative ACL rupture in dogs because bacteria-associated stifle synovitis is common and because dogs are one of the few higher mammals where the MHC genomic organization is well understood. (IMGT/HLA and IMGT/MHC sequence databases at http://www.ebi.ac.uk/imgt/hla and http://www.ebi.ac.uk/imgt/mhc; and, Robinson et al., IPD—the immuno polymorphism database, Nucleic Acids Res 2005, 33:D523-D526). Spontaneous canine diseases model equivalent disease in humans, such as inflammatory, autoimmune and neoplastic disease.

Dogs are also an important model for studying experimental organ transplantation and spontaneous small joint polyarthritis in dogs having MHC class II susceptibility. (Ollier et al., Dog MHC alleles containing the human RA shared epitope confer susceptibility to canine rheumatoid arthritis, Immunogenetics 2001, 53:669-673). Many dog breeds have also been in existence for less than 200 years. Severe selection pressure has also increased MHC class II homozygosity and reduced variation in MHC class II alleles within individual dog breeds. (Kennedy et al., 2002).

Thus, the susceptibility of different dog breeds to inflammatory disease triggered by bacteria may be different, and inflammatory/degenerative ACL rupture is predisposed in certain breeds, such as Labradors and Newfoundlands. (Whiteheair et al., Epidemiology of cranial cruciate ligament ruptured in dogs, J Am Vet Med Assoc 1993, 203:1016-1019; and, Duval et al., Breed, sex and body weight as risk factors for rupture of the cranial cruciate ligament in young dogs, J Am Vet Med Assoc 1999, 215:811-814).

In 2005, more than 500 published papers from the scientific and medical communities focused on the problem of inflammatory arthritis. Such widespread attention underscores the significant interest in mechanisms related to chronic joint inflammation. In spite of that widely appreciated importance, there remains a general lack of understanding of any human-microbe ecology. (Gerard et al., Chromosomal DNA from a variety of bacterial species is present in synovial tissue from patients with various forms of arthritis, Arthritis Rheum 2001, 44:1689-1697). That critical knowledge gap centers on the key bacteria-related factors that trigger and maintain organ-specific inflammatory responses.

In the early 1980's, it was discovered that Lyme disease is caused by a spirochaete infection. (Steere et al., Lyme arthritis: an epidemic of oligoarticular arthritis in children and adults in three Connecticut communities, Arthritis Rheum 1977, 20:7-17; and, Burgdorfer et al., Lyme disease—a tick-borne spirochaetosis? Science 1982, 216:1317-1319). Since then, considerable effort has been devoted to understanding how bacteria trigger the immune system and promote chronic disease. It is now recognized that highly complex mechanisms are involved concerning interaction between bacteria (such as Borrelia burgdorferl) and the immune system that promotes joint inflammation. (Batsford et al., Outer surface lipoproteins of Borrelia burgdorferi vary in their ability to induce experimental joint injury, Arthritis Rheum 2004, 50:2360-2369).

### SUMMARY OF THE INVENTION

One aspect of the invention is a method of diagnosing persistent, chronic synovitis in the joint of a mammal comprising the steps or acts of providing a test sample comprising synovial fluid, cell or tissue from the joint of a mammal; quantifying the concentration of one or more protein or mRNA biomarkers being cathepsin K, MMP-2, MMP-9, cathepsin S, TRAP, invariant chain, CD4⁺ T-lymphocytes, CD8⁺ T-lymphocytes, CD44⁺ mononuclear cells, Toll-like receptor-2, or Toll-like receptor-9, or a combination thereof; and, comparing the concentration of the one or more biomarkers from the test sample to a corresponding biomarker concentration in an internal control sample; wherein a statistically significant elevated concentration of the one or more biomarkers in the test sample indicates that the mammal's joint is diseased with persistent, chronic synovitis.

Another aspect of the invention is a method of diagnosing progressive joint degradation in the joint of a mammal comprising the steps or acts of providing a test sample comprising synovial fluid, cell or tissue from the joint of a mammal; quantifying the concentration of one or more protein or mRNA biomarkers being cathepsin K, MMP-2 MMP-9, cathepsin S, TRAP, invariant chain, CD4⁺ T-lymphocytes, CD8⁺ T-lymphocytes, CD44⁺ mononuclear cells, Toll-like receptor-2, or Toll-like receptor-9, or a combination thereof; and, comparing the concentration of the one or more biomarkers from the test sample to a corresponding biomarker concentration in an internal control sample; wherein a statistically significant elevated concentration of the one or more biomarkers in the test sample indicates that the mammal's joint is diseased with progressive joint degradation.

As regards the diagnostic methods of the present invention, the internal control sample comprises PMBC. Preferably, the elevated concentration is statistically significant at P<0.05 to both the internal control and an external control from a healthy individual.

Another aspect of the invention is a method of diagnosing synovitis and progressive joint degradation of a mammal comprising the steps or acts of providing a test sample comprising synovial fluid, cell or tissue from the joint of a mammal and detecting the presence of bacterial DNA, particularly mixtures of bacterial DNA. The mammal may be a dog or a human. The synovial fluid, cell or tissue may contain bacterial DNA generated by mixtures of environmental bacterium, including but not limited to Borrelia burgdorferi, Stenotrophomonas maltophilia, uncultured Eubacterium, Rhizobium radiobacter, Ralstonia solanacearum, uncultured beta Proteobacterium, Achromobacter xylosoxidans, uncultured Oxalobacterium, Corynebacterium glutamicum, Rhizobium galegae, Gordonia terrae, Acinetobacter calcoaceticus, Pseudomonas putida, uncultured Gemmatiomonadates bacterium or uncultured Burkholderia.

Another aspect of the invention is a method of diagnosing genetic predisposition to inflammatory arthritis comprising the steps or acts of providing a test sample containing host DNA and determining the HLA-DRB1 or DLA-DRB1 allele. In an exemplary embodiment, the dog has major histocompatibility complex class II alleles, particularly but not limited to DLA-DRB1*0102 and DLA-DRB1*1502.

In an exemplary embodiment, the protein or mRNA biomarker is cathepsin K, MMP-2, MMP-9, cathepsin S, TRAP, invariant chain, Toll-like receptor-2, Toll-like receptor-9, or a combinations thereof, and the concentration of the one or more biomarkers are measured using ELISA, or RNAzol B methodology including quantitative RT-PCR.

In another exemplary embodiment, the biomarker is CD4, CD8, CD44, MHC class II, CD11b, bacterial peptidoglycan, CD25, or combinations thereof, and the expression of one or more of the biomarkers is measured using flow cytometry.

In another exemplary embodiment, the biomarker is CD4⁺ T-lymphocytes, CD8⁺ T-lymphocytes, CD44⁺ mononuclear cells, Toll-like receptor-2, Toll-like receptor-9, or combinations thereof, wherein the concentration of the one or more biomarkers are measured using flow cytometry.

In another exemplary embodiment, the biomarker is CD4⁺ T-lymphocytes, CD8⁺ T-lymphocytes, CD44⁺ mononuclear cells, peptidoglycan⁺ mononuclear cells, Toll-like receptor-2, Toll-like receptor-9, or combinations thereof, wherein the concentration of the one or more biomarkers are measured using an enzyme-linked immunosorbent assay.

In another exemplary embodiment, the expression of cathepsin K is elevated around 167-fold, compared with the internal PBMC control.

In another exemplary embodiment, the expression of MMP-9 is elevated around 4053-fold, compared with the internal PBMC control.

In another exemplary embodiment, the expression of cathepsin S is elevated around 60-fold, compared with the internal PBMC control.

In another exemplary embodiment, the expression of TRAP is elevated around 51-fold, compared with the internal PBMC control.

In another exemplary embodiment, the expression of invariant chain is elevated around 15-fold, compared with the internal PBMC control.

In another exemplary embodiment, the expression of MMP-9 is elevated around 196-fold, compared with stifle joint fluid from healthy dogs.

In another exemplary embodiment, the expression of TRAP is elevated around 35-fold, compared with stifle joint fluid from healthy dogs.

In another exemplary embodiment, the expression of invariant chain is elevated around 14-fold, compared with stifle joint fluid from healthy dogs.

Another aspect of the invention is a method of diagnosing synovitis in the joint of a mammal comprising the steps or acts of providing a test sample comprising synovial fluid, cell or tissue from the joint of a mammal, and, detecting the presence of bacterial DNA or mixtures of bacterial DNA. The mammal may be a dog or a human.

In an exemplary embodiment, the bacterial DNA is generated by one or more of Borrelia burgdorferi, Stenotrophomonas maltophilia, uncultured Eubacterium, Rhizobium radiobacter, Ralstonia solanacearum, uncultured beta Proteobacterium, Achromobacter xylosoxidans, uncultured Oxalobacterium, Corynebacterium glutamicum, Rhizobium galegae, Gordonia terrae, Acinetobacter calcoaceticus, Pseudomonas putida, uncultured Gemmatiomonadates bacterium, and, uncultured Burkholderia.

Another aspect of the invention is a method of diagnosing genetic predisposition to inflammatory arthritis in a host dog comprising the steps or acts of providing a test sample containing host dog DNA, and, determining the HLA-DRB1 or DLA-DRB1 allele.

In an exemplary embodiment, the host dog has major histocompatibility complex class II alleles.

In another exemplary embodiment, the major histocompatibility complex class II alleles are a member selected from the group consisting of DLA-DRB1*0102 and DLA-DRB1*1502.

Another aspect of the invention is a method of treating 16S rRNA PCR-positive joints in a mammal comprising administering to the mammal a therapeutic amount of a tetracycline. Preferably, the tetracycline is doxycycline. The mammal may be a dog or a human.

### BRIEF DESCRIPTION OF THE DRAWINGS

FIGS. 1-3 are graphs showing the proportion of CD4⁺ helper T-lymphocytes (FIG. 1) and CD8⁺ cytotoxic T-lymphocytes (FIG. 2) in joint and PBMC populations in dogs with chronic stifle synovitis/degenerative ACL rupture, whereby large numbers of CD4⁺ and CD8⁺ T lymphocytes were detected in the stifle joints of affected dogs, whereby a large proportion of the mononuclear cells within the stifle joints also expressed the activation marker CD44 (FIG. 3), whereby the boxes represent 25th and 75th percentiles, and whereby outliers are also plotted.

FIGS. 4-8 are graphs showing relative gene expression being cathepsin K expression (FIG. 4), MMP-9 expression (FIG. 5), cathepsin S expression (FIG. 6), TRAP expression (FIG. 7), and invariant chain expression (FIG. 8) in the stifle synovial fluid normalized to PBMC as an internal control, whereby 18S rRNA was used as the housekeeping gene, whereby # indicates a significant difference from the internal PBMC controls (P<0.05), whereby boxes represent median, and the 25th and 75th percentiles, whereby whiskers represent 10th and 90th percentiles, whereby outliers are also plotted, and whereby data were summarized from 32 ACL-ruptured dogs, 8 normal dogs, and 9 dogs with osteoarthritis (OA).

FIG. 9 shows a representative flow cytometry plots from a 7-month-old healthy female Beagle with a normal stifle and an intact cranial cruciate ligament (CCL), and a 3-year-old neutered male Beagle affected with oligoarthritis and associated degenerative CCL rupture (CCLR). Notice that large populations of CD4+ and CD8+ T lymphocytes were detectable in the synovium of the dog with inflammatory stifle arthritis.

FIGS. 10-15 Proportion and numbers of CD4+ (FIGS. 10 and 11) and CD8+ (FIGS. 12 and 13) T lymphocytes in joint and PBMC populations, whereby mononuclear cell populations in synovial tissue from dogs with oligoarthritis and associated degenerative cranial cruciate ligament rupture (CCLR) contain many CD4+ and CD8+ T lymphocytes, whereby a large proportion of joint mononuclear cells in dogs with oligoarthritis also express the activation marker CD44 (FIGS. 14 and 15), whereby data are summarized from 23 dogs with stifle oligoarthritis, whereby PBMC were collected from 11 healthy dogs, whereby joint tissues were also collected from 6 of these healthy dogs, whereby CD44+ cells were quantified in 7 dogs with oligoarthritis and 8 healthy dogs, whereby boxes represent median and the 25th and 75th percentiles, whereby whiskers represent 10th and 90th percentiles, whereby mean is represented by a dashed line, and whereby significant differences between tissue types and between groups are indicated.

FIGS. 16-19 show a month-by-month plot of the proportion of canine stifle joints affected with degenerative cranial cruciate ligament rupture and containing panbacterial 16S rRNA in 2006 (FIG. 16), Borrelia burgdorferi OspA (FIG. 17), Stenotrophomonas maltophilia 23S rRNA DNA sequences (FIG. 18), and the proportion of joints being PCR-positive for bacteria (FIG. 19), whereby both 16S rRNA and OspA detection varied significantly according to the time of the year.

FIGS. 20-26 show relative expression of cathepsin K (FIG. 20), cathepsin S (FIG. 21), tartrate-resistant acid phosphatase (TRAP) (FIG. 22), matrix metallporoteinase-9 (MMP-9) (FIG. 23), invariant chain (li) (FIG. 24), toll-like receptor-2 (TLR-2) (FIG. 25), and TLR-9 (FIG. 26) in synovial fluid cells collected from dogs with stifle oligoarthritis and associated degenerative cranial cruciate ligament (CCL) rupture, stifle synovial fluid of healthy dogs, and canine joints affected with other forms of arthritis, whereby gene expression was normalized to peripheral blood mononuclear cells (PBMC) as an internal control using the ΔΔCₜ method, whereby 18S rRNA was used as the housekeeping gene, whereby # indicates a significant difference from the internal PMBC controls (P<0.05), whereby boxes represent median and the 25^{th} and 75^{th} percentiles, whereby whiskers represent 10^{th} and 90^{th} percentiles, whereby outliers are plotted, and whereby significant differences between groups are as indicated.

FIG. 27 shows a photograph of the synovial vasculature viewed arthroscopically during treatment of a 10-year-old neutered male Siberian Husky with bilateral stifle oligoarthritis and associated degenerative cranial cruciate ligament rupture.

FIG. 28 shows a photomicrograph of synovial membrane from a 4-year-old spayed female Labrador cross with lameness, stifle oligoarthritis and a stable stifle, whereby effusion and osteophyte formation was evident radiographically, whereby the cranial cruciate ligament was grossly normal at surgery, whereby infiltration of the synovial intima and the subintimal tissues with mononuclear inflammatory cells is evident, and whereby H&E frozen section is also shown.

FIGS. 29 and 30 show representative flow cytometry plots for dogs, whereby there is a higher proportion of interferon gamma⁺ CD4⁺ cells in the stifle as compared to PBMC, and whereby T lymphocytes within stifle synovial fluid of CCLR dogs were studied.

FIGS. 31 and 32 show representative flow cytometry plots for dogs with CCLR, whereby very few B lymphocytes were detected in the stifle synovial joint fluid.

FIGS. 33 and 34 show representative flow cytometry plots for dogs with elbow arthritis/dysplacia, which is an example of osteoarthritis clinically as compared with inflammatory arhtritis, whereby there are substantial amounts of monocyte macrophages and APC's in synovial fluid, specifically CD11b⁺ Class II⁺ cells in joint fluid.

FIGS. 35 and 36 show representative flow cytometry plots for dogs with CCLR and stifle arthritis, whereby relatively fewer class II⁺ CD11b⁺ cells are present in the synovial joint fluid.

FIGS. 37-40 show a photomicrograph of the synovial membrane (FIG. 37) and relevant radiographs (FIGS. 38-40) of a 4-year-old NF labrador cross with stifle oligoarthritis and a stable contralateral stifle lameness (which is not typically very responsive to NSAID's), and whereby the CCL was grossly normal during TPLO surgery, whereby a second TPLO surgery was performed a year later.

FIGS. 41 and 42 show representative flow cytometry plots for dogs with cruciate rupture, whereby the proportion of peptidoglycan⁺ class II⁺ cells in joint fluid is higher than in PBMC suggesting that bacterial material is being presented by antigen-presenting cells to T lymphocytes.

FIG. 43 shows a representative flow cytometry plots from a dog with a CCL rupture, the stilfe stifle joint fluid contains a significant amount of CD⁺ INF-gamma⁺ T lymphocytes.

### DETAILED DESCRIPTION OF THE PREFERRED EMBODIMENTS

Rupture of the cranial cruciate ligament is one of the most important orthopaedic conditions of dogs and leads to a large economic burden on dog owners. Despite the importance of this arthropathy, the mechanism that leads to cruciate rupture is not understood. Current surgical treatments, such as tibial plateau leveling osteotomy and tibial tuberosity advancement are widely performed. However, such procedures do not restore weight-bearing to normal. (Conzemius M G et al., Effect of surgical technique on limb function after surgery for rupture of the cranial cruciate ligament in dogs, J Am Vet Med Assoc 2005, 226:232-236).

Furthermore, surgical treatment does not ameliorate progression of arthritis over time based on radiographic assessment and analysis of synovial biomarkers (Girling S L et al., Use of biochemical markers of osteoarthritis to investigate the potential disease-modifying effects of tibial plateau leveling osteotomy, J Small Anim Pract 2006, 47:708-714; Lineberger J A et al., Comparison of radiographic arthritic changes associated with two variations of tibial plateau leveling osteotomy, Vet Comp Orthop Traumatol 2005, 18:13-17; and, de Bruin T et al., Evaluation of anticollagen type I antibody titers in synovial fluid of both stifle joints and the left shoulder joint of dogs with unilateral cranial cruciate disease, Am J Vet Res 2007, 68:283-289).

A likely explanation for these findings is that surgical procedures (such as tibial osteotomy) only provide treatment for dynamic instability of the stifle. However, such procedures do not treat passive instability or other relevant stifle pathology. Fundamental understanding of the disease mechanism for degenerative cruciate rupture may hold the promise of further innovation in the overall management of dogs with stifle arthritis. Following is a review what is known about the cruciate rupture disease mechanism.

Clinical Features. Cruciate rupture as a consequence of traumatic injury represents a minority of cases, and it is typically associated with avulsion of a bone fragment at the proximal or distal ligament attachment site. (Hayashi K et al., Cranial cruciate ligament pathophysiology in dogs with cruciate disease: A review, J Am Anim Hosp Assoc 2004, 40:385-390). The majority of dogs with degenerative mid-substance rupture of the cranial cruciate ligament have a history of low-grade prodromal lameness. Careful palpation of the stifle may reveal either a stable stifle or very mild instability.

Subsequent development of more severe lameness is typically associated with more obvious palpable stifle instability. Prodromal lameness may persist for some time and is often not recognized by dog owners. Clinical and radiographic signs of arthritis and effusion are often present bilaterally at the time of diagnosis. (Doverspike M et al., Contralateral cranial cruciate ligament rupture: Incidence in 114 dogs, J Am Anim Hosp Assoc 1993, 29:167-170).

Synovial fluid biomarkers of arthritis and joint degradation are also elevated bilaterally in affected dogs. (Steffey M A et al., The potential and limitations of cartilage-specific (V+C)(−) fibronectin and cartilage oligomeric matrix protein as osteoarthritis biomarkers in canine synovial fluid, Osteoarthr. Cartil 2004, 12:818-825).

In one study of dogs with unilateral cruciate rupture and radiographic abnormalities in the contralateral stifle, 55% of dogs developed contralateral cruciate rupture an average of 16 months after initial diagnosis. (Doverspike M. et al., 1993). At surgery, typical findings include palpable instability, indicating complete tearing of the ligament, and osteophyte formation. It is common for a small portion of the cruciate ligament to retain anatomic attachments to the femur and tibia. Resorption of the ruptured ends of the ligament may also be seen. Quantitative histological examination of ruptured cruciate tissue suggests progressive mechanical overload of ligament tissue. (Hayashi K et al., Histologic Changes in Ruptured Canine Cranial Cruciate Ligament, Vet Surg 2003, 32:269-277; and, Hayashi K et al., Evaluation of ligament fibroblast viability in ruptured cranial cruciate ligament of dogs. Am J Vet Res 2003, 64:1010-1016).

Taken together, these findings suggest that degenerative cruciate rupture is a consequence of a pre-existing arthropathy, whereby ligament rupture occurs progressively over time such that (in the early phase of the condition) the stifle is stable. Eventually, as the arthropathy progresses, complete rupture of the cranial cruciate occurs leading to the development of stifle instability. It is common for remnants of disrupted ligament to retain attachments to the femur and tibia.

Joint Pathology. Moderate to severe synovitis is typically seen, but usually without full thickness cartilage loss. Histologic studies have shown the synovium contains B and T lymphocytes, activated TRAP⁺ macrophages, MHC class II⁺ dendritic cells, and plasma cells. (Lemburg A K et al., Immunohistochemical characterization of inflammatory cell populations and adhesion molecule expression in synovial membranes from dogs with spontaneous cranial cruciate ligament rupture, Vet Immunol Immunopathol 2004, 97:231-240; Klocke N W et al., Detection of synovial macrophages in the joint capsule of dogs with naturally occurring rupture of the cranial cruciate ligament, Am J Vet Res 2005, 66:493-499; and, Muir P et al., Collagenolytic gene expression in cranial cruciate ligament and stifle synovial fluid in dogs with cranial cruciate ligament rupture, Vet Surg 2005, 34:482-490).

Flow cytometry has confirmed that the synovium contains large populations of both CD4⁺ T helper lymphocytes and CD8⁺ cytotoxic T lymphocytes. Up-regulation of expression of matrix turnover genes and antigen-specific immune response genes is associated with development of synovial inflammation. (Muir P et al., Detection of DNA from a range of bacterial species in the stifle joints of dogs with inflammatory stifle arthritis and associated degenerative anterior cruciate ligament rupture, Microb Pathog 2007, 42:47-55; and, Muir P et al., Expression of immune response genes in the stifle joint of dogs with oligoarthritis and degenerative cranial cruciate ligament rupture, Vet Immunol Immunopathol 2007, epub 2007).

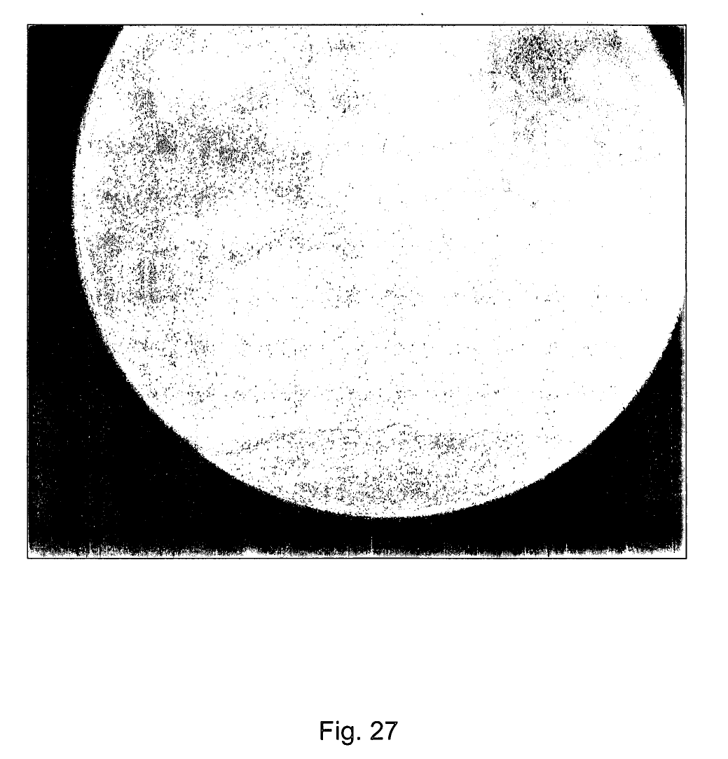

As shown in FIG. 27, the synovial capillary vasculature typically has a tortuous appearance. Taken together, these data suggest that the cruciate rupture arthropathy is an oligoarthritis, which is defined as an inflammatory arthritis affecting ≦4 joints. The cell populations present within the synovium suggest that synovial inflammation is driven by antigen-specific immune responses. The vascular pattern within the synovium is similar to that which is typically found in certain types of rheumatic disease in human beings, such as reactive arthritis. (Reece R J et al., Distinct vascular patterns of early synovitis in psoriatic, reactive, and rheumatoid arthritis, Arthritis Rheum 1999, 42:1481-1484). The synovitis which develops in reactive arthritis is a consequence of dysregulation of immune responses by bacterial antigens.

Relationship of synovitis to stifle instability. The classical paradigm for development of stifle arthritis in dogs with cruciate rupture suggests that synovitis is largely a consequence of cruciate rupture and development of stifle instability. This is supported by the fact that transection of the cranial cruciate ligament in experimental dogs leads to synovitis. (Lipowitz A J et al., Synovial membrane changes after experimental transection of the cranial cruciate ligament in dogs, Am J Vet Res 1985, 46:1166-1170).

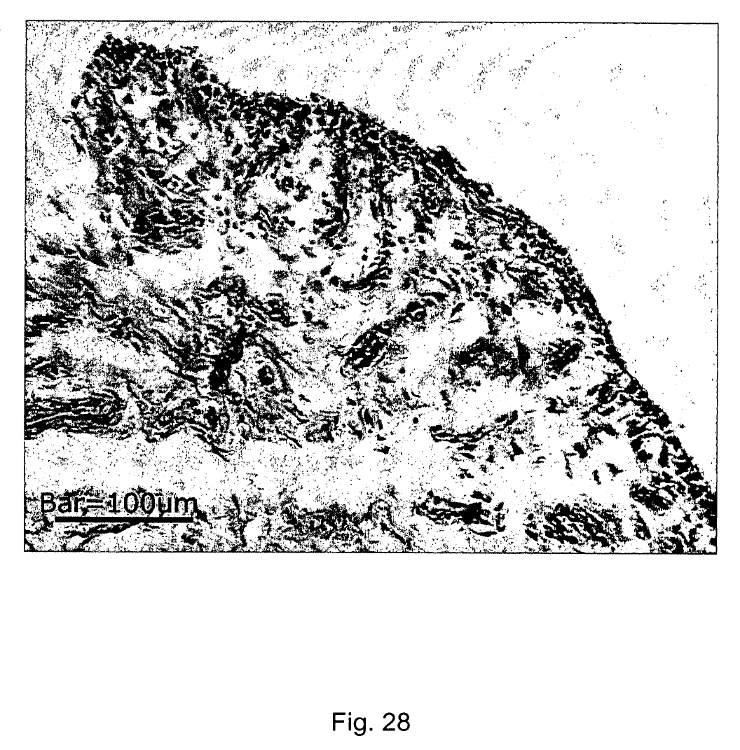

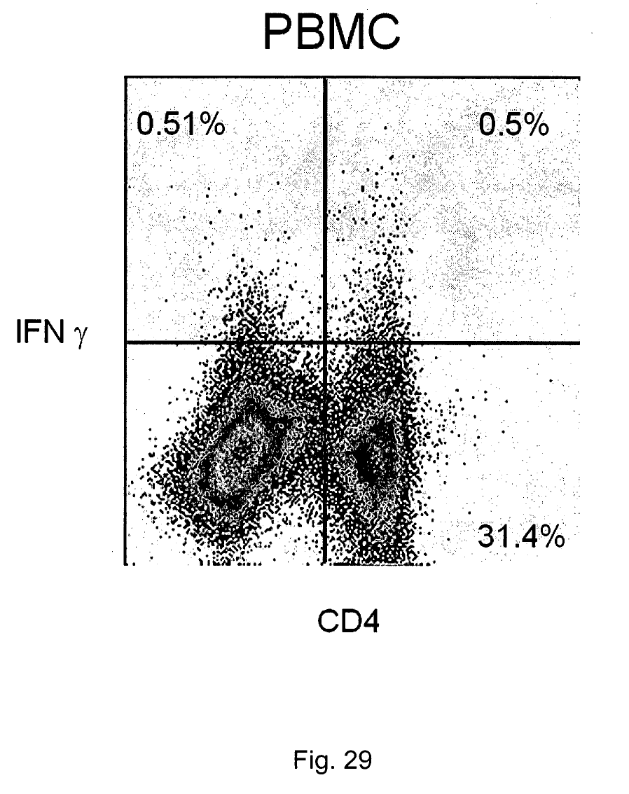

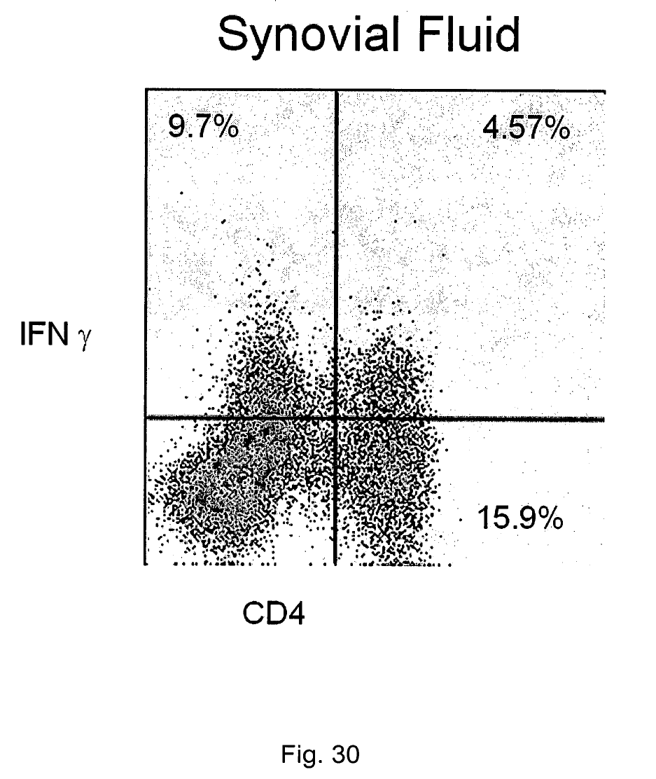

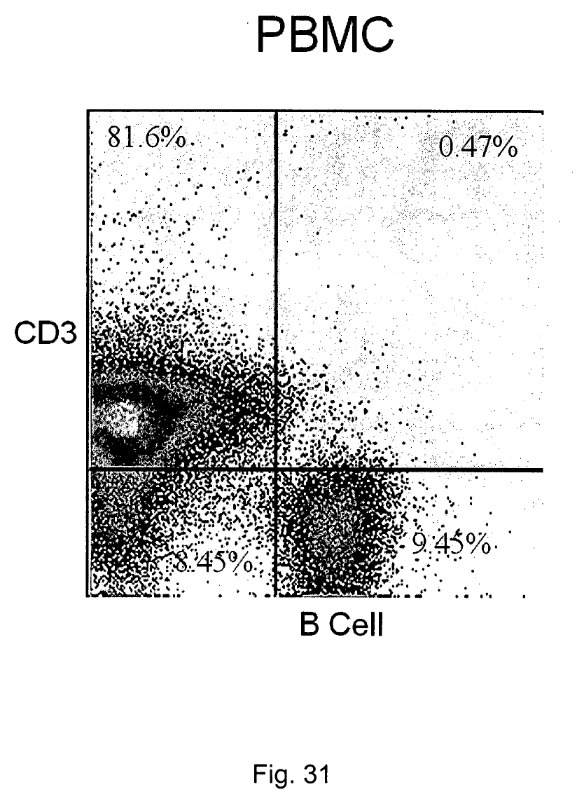

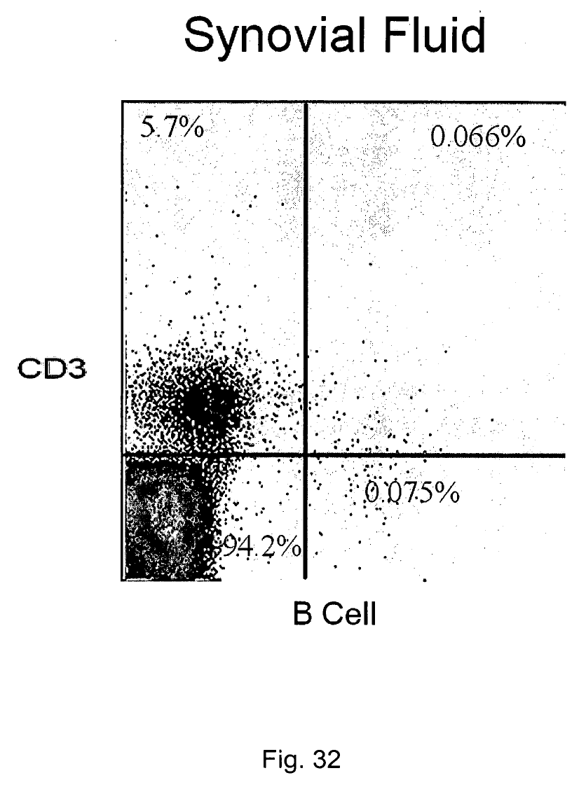

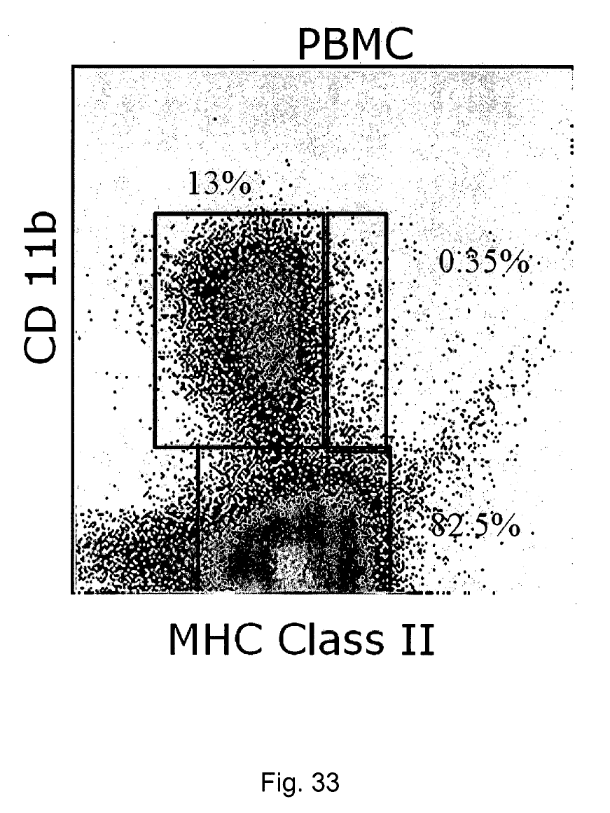

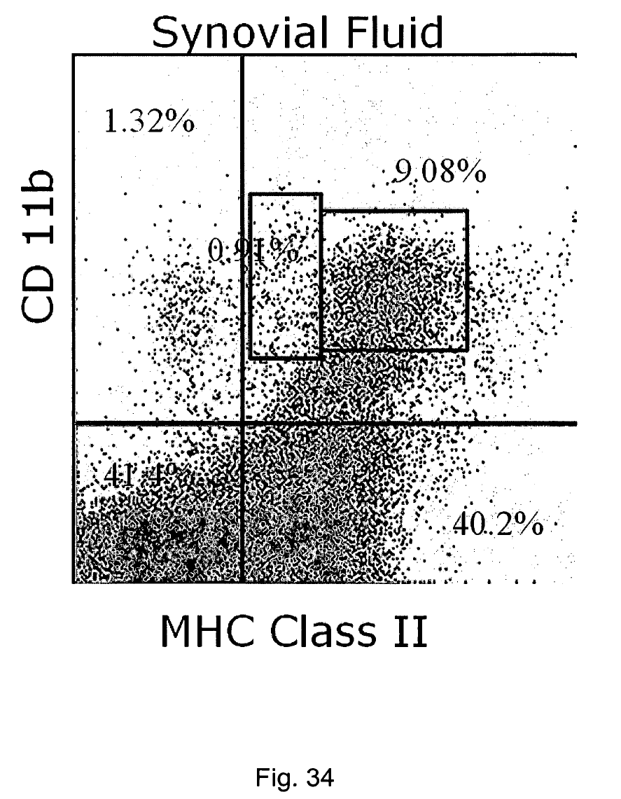

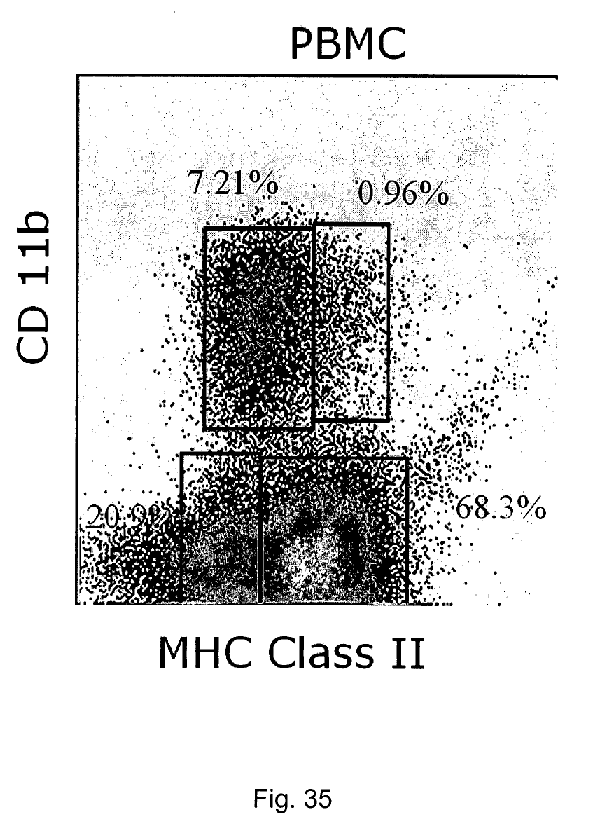

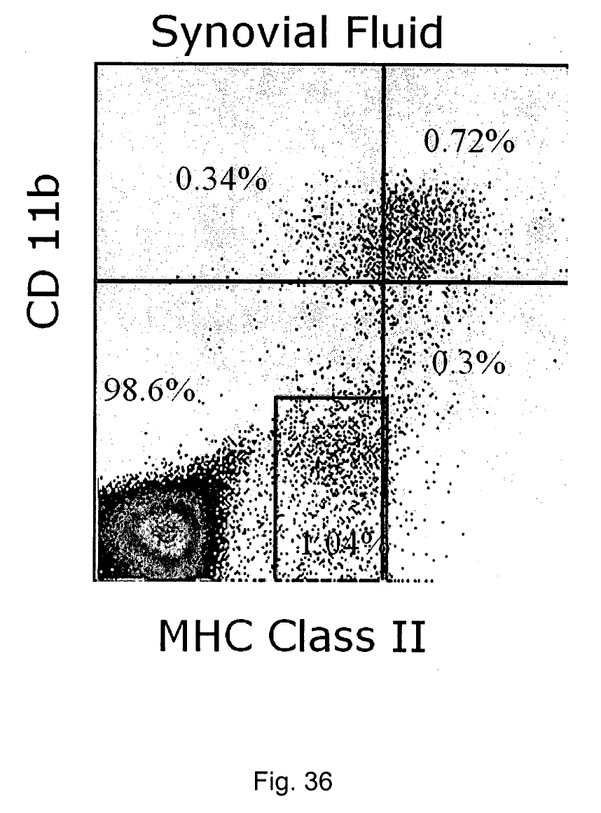

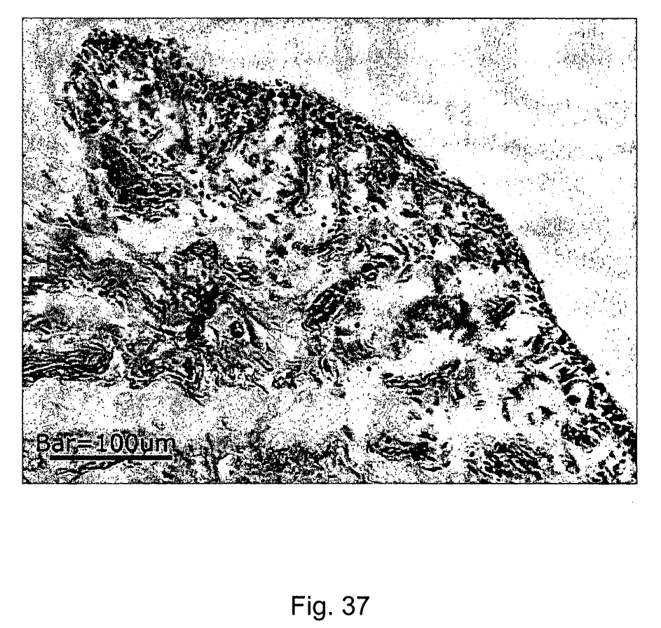

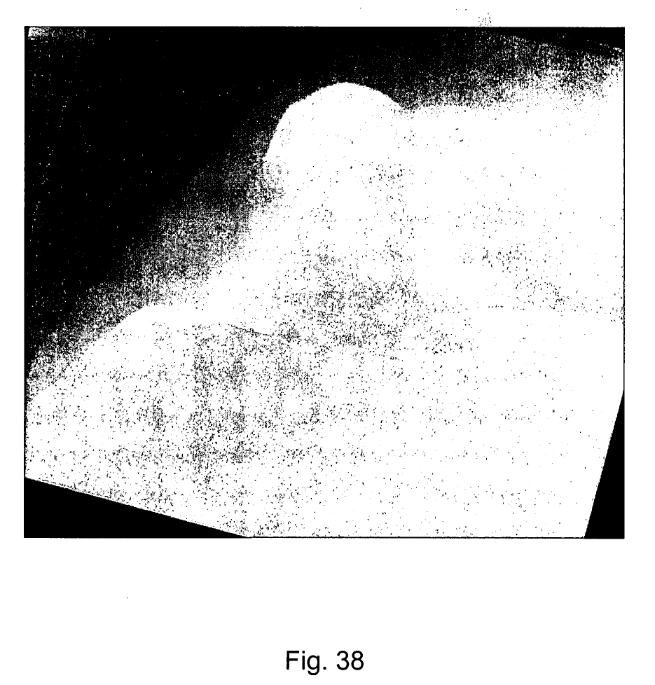

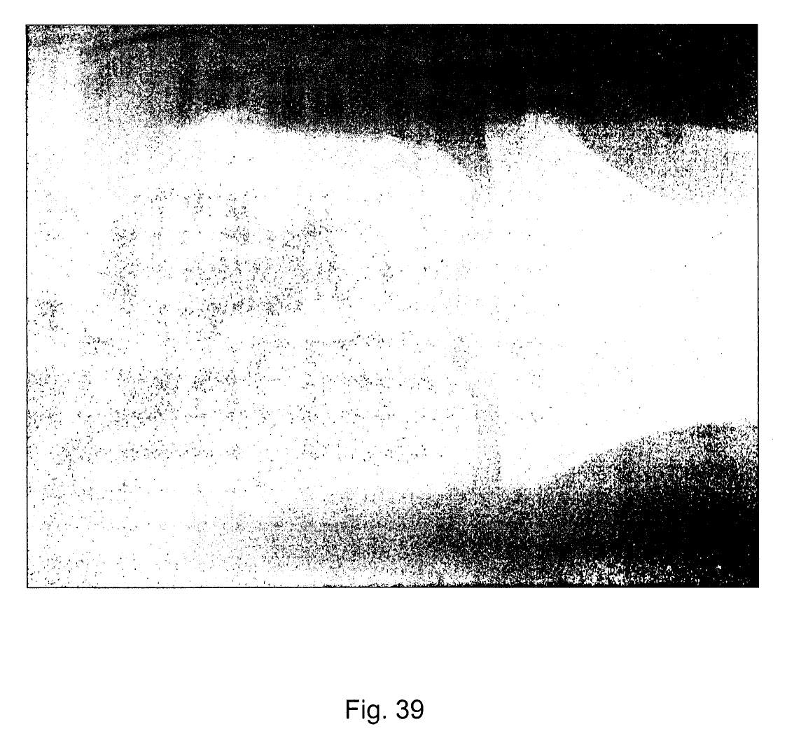

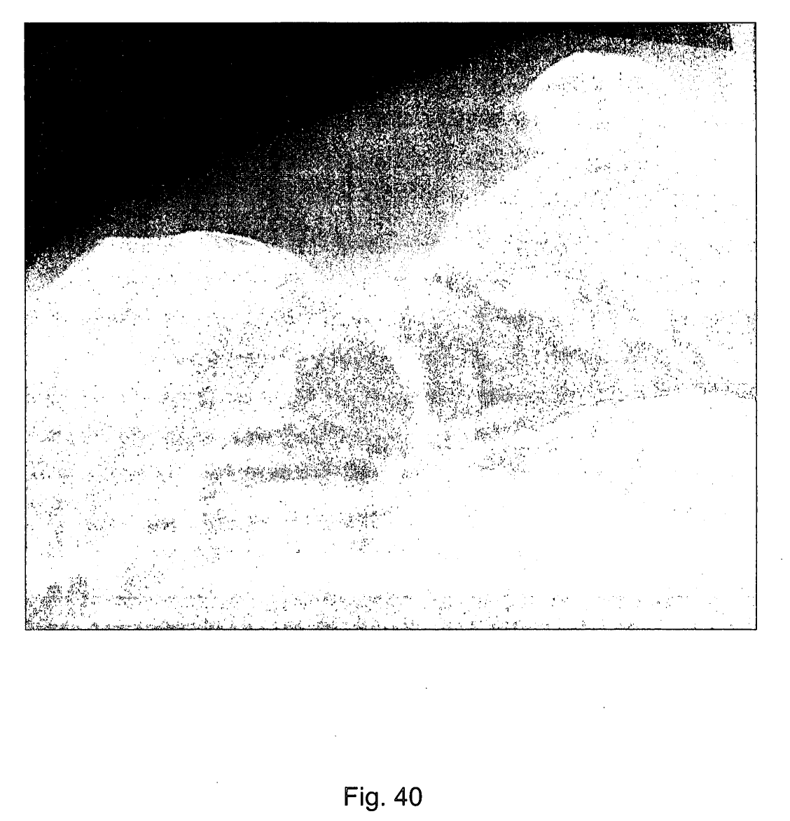

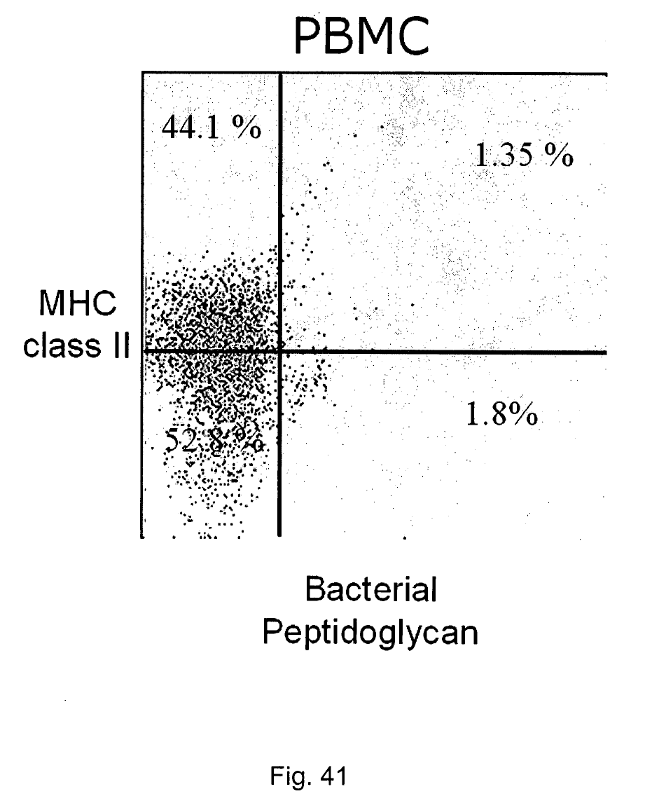

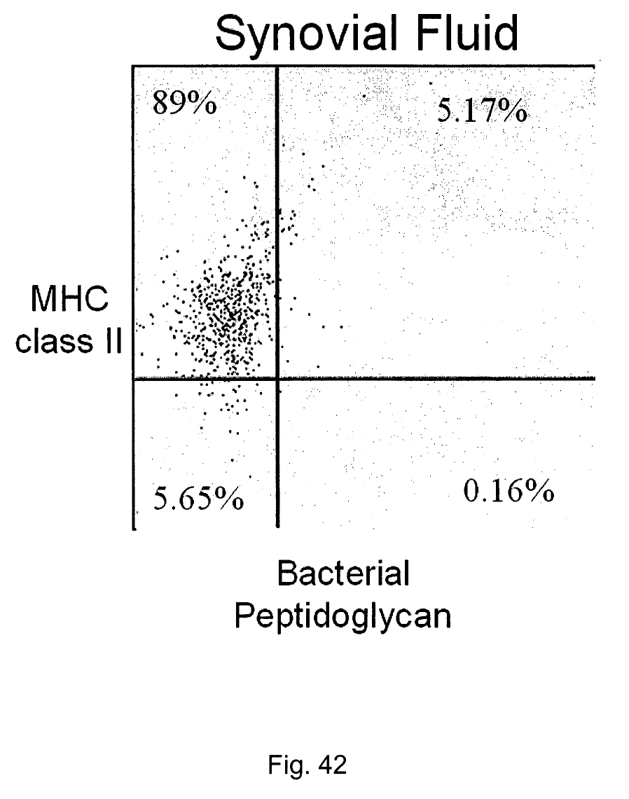

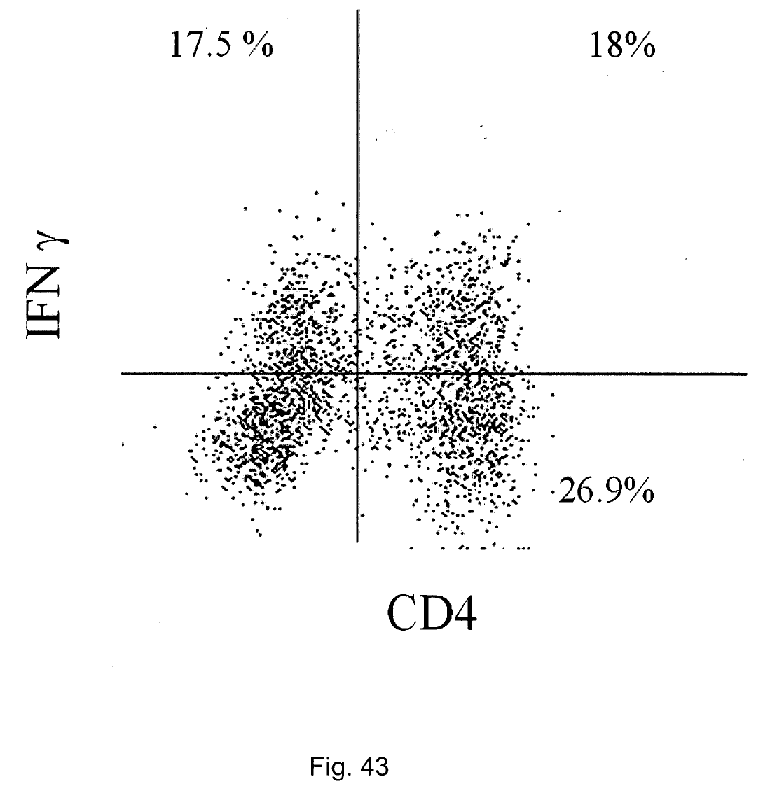

However, in dogs with the prodromal phase of the disease, mild lameness, and a stable stifle joint synovitis is also found as shown in FIG. 28 together with elevation of arthritis biomarkers in synovial fluid. (Myers S L et al., Synovitis and osteoarthritic changes in canine articular cartilage after anterior cruciate ligament transection: Effect of surgical hemostasis, Arthritis Rheum 1990, 33:1406-1415); Doverspike et al., 1993; Steffey et al., 2004; and, de Bruin et al., 2007).

Taken together, these data suggest that the classical paradigm between synovitis and cruciate rupture needs to be updated and that the dogs with prodromal lameness, stable stifles and incipient cruciate rupture are typically affected with an oligoarthritis.

Next is a discussion of the key factors that promote degenerative cruciate rupture. Several different factors have been proposed as contributing to the development of degenerative cruciate rupture including: Stifle morphology (Odders J W et al., Sequential measurements of the tibial plateau angle in large-breed growing dogs, Am J Vet Res 2004, 65:513-518); altered ligament composition and metabolism in predisposed breeds (Comerford E J et al., Metabolism and composition of the canine anterior cruciate ligament relate to differences in stifle joint mechanics and predisposition to ligament rupture, J Orthop Res 2005, 23:61-66); narrowing of the femoral intercondylar notch width (Comerford E J et al., Distal femoral intercondylar notch dimensions and their relationship to composition and metabolism of the canine anterior cruciate ligament, Osteoarthr Cartil 2006, 14:273-278); age-related cruciate ligament degeneration (Vasseur P B et al., Correlative biomechanical and histologic study of the cranial cruciate ligament in dogs, Am J Vet Res 1985, 46:1842-1854); and synovitis (Muir et al., 2005).

Studies of ligament biochemistry have shown that progressive rupture of the cruciate is associated with up-regulation of matrix turnover, apoptosis and necrosis of ligament fibroblasts, and fragmentation of matrix collagen. (Hayashi et al., 2003; Volk S W et al., Gelatinase activity in synovial fluid and synovium obtained from healthy and osteoarthritic joints of dogs, Am J Vet Res 2003, 64:1225-1233; Muir et al., 2005; Muir P et al., Collagen fragmentation in ruptured canine cranial cruciate ligament explants, Vet J 2006, 172:121-128; and, Gyger O et al., Detection and distribution of apoptotic cell death in normal and diseased canine cranial cruciate ligaments, Vet J epub 2007).

Recent studies of the microvascular architecture of the cranial cruciate ligament and the overlying synovial intima suggest that the ligament derives most of its nutrition from synovial fluid. (Kobayashi S et al., Microvascular system of the anterior cruciate ligament in the dog, J Orthop Res 2006, 24:1509). Therefore, synovitis and associated release of pro-inflammatory cytokines and matrix degrading enzymes into synovial fluid may have a profound effect on ligament collagen metabolism.

As synovitis develops in affected dogs, the adjacent ligament tissue also becomes heavily infiltrated with inflammatory cells, including activated macrophages expressing proteases. (Muir et al., 2005). These concepts are supported by experimental studies in which induction of stifle synovitis caused significant degradation in the tensile strength of the cranial cruciate ligament to 29% of control by 6 weeks. (Goldberg V M et al., The influence of an experimental immune synovitis on the failure mode and strength of the rabbit anterior cruciate ligament, J Bone Joint Surg 1982, 64A:900-906).

In control stifles, the typical mode of cruciate failure during tensile biomechanical testing involved fracture of bony attachment sites. With induction of synovitis, the most common mode failure was mid-substance rupture. (Goldberg et al., 1982). Taken together, these data suggest that the stifle inflammatory arthritis that precedes development of stifle instability is a key factor promoting progressing weakening of the cranial cruciate ligament and eventual mid-substance rupture. Genetic factors may explain breed-predisposition to the arthropathy.

Following is a discussion of the causes stifle oligoarthritis in affected dogs. The immunologic trigger for canine stifle oligoarthritis is not known. This arthritis may be a form of autoimmune disease. Exposure of collagen neoepitopes and development of collagen autoantibodies in synovial fluid may promote stifle degradation.

However, the development of collagen autoantibodies is not a primary causative factor. (de Bruin et al., 2007). Translocation of bacterial material to the stifle has also been implicated as a causative factor. The presence of bacterial DNA within the stifle is associated with the cruciate arthropathy as compared to normal stifles and stifles from dogs with experimentally-induced cruciate rupture. (Muir et al., 2007). In human rheumatic disease, bacterial material commonly triggers inflammatory arthritis. This phenomenon often affects the knee joint. (Schumacher H R, How micro-organisms are handled to localize to joints and within joints, Scand J Rheumatol 24(Suppl 101):199-202, 1995).

Important examples of this type of rheumatic disease include reactive arthritis, which is triggered by gastrointestinal or urogenital infection with a bacterial pathogen. (Saxena S et al., Outer membrane protein of Salmonella is the major antigenic target in patients with Salmonella induced reactive arthritis, J Rheumatol 2005, 32:86-92). Treatment-resistant chronic Lyme arthritis can also develop in human patients who fail to respond to antibiotic therapy. Specific MHC class II alleles confer susceptibility enabling Borrelia burgdorferi material to trigger dysregulation of host immunity and development of a chronic inflammatory arthritis that often affects the knee. (Shin J J et al., High levels of inflammatory chemokines and cytokines in joint fluid and synovial tissue throughout the course of antibiotic-refractory Lyme arthritis, Arthritis Rheum 2007, 56:1325-1335).

Taken together, recent research suggests that translocation of bacteria to the stifle joint is a common event in the dog. The presence of bacterial material within the stifle may be a key pro-inflammatory factor triggering chronic synovitis in susceptible dogs. Host-pathogen interactions likely involve genetic predisposition.

Past research and studies clearly demonstrates a long felt and unmet need to identify whether stifle inflammatory arthritis and degenerative ACL ruptures develop preferentially in dogs having specific MHC class II haplotypes. There also clearly exists another long felt and unmet need to understand whether specific MHC class II haplotypes are significantly associated with detectable bacterial DNA within synovial stifle tissue.

Without being bound to any theory, it is hypothesized that the immunological component of ligament disease is due to bacterial infection. To support that hypothesis, a PCR-based assay was used to detect and speciate bacterial DNA in synovial membrane, cranial cruciate ligament, and synovial fluid. The present invention is supported by a showing that the stifle joints of 35-47% of dogs having cruciate ligament rupture contain bacterial DNA from mixtures of bacteria (most commonly the bacteria Stenotrophomonas maltophilia and Borrelia burgdorferi indicating infection) whereas dogs without any such rupture were not similarly infected.

It is further theorized that a genetic component is involved that predisposes some mammals to developing arthritis and ligament rupture resulting from such bacterial infections. Polymorphisms in the HLA-DRB1 gene in humans affects MHC class II antigen processing. Specific alleles may alter the risk of autoimmune responses such as rheumatoid arthritis. Similar DLA-DRB1 polymorphisms also exist in dogs. The invention also provides a canine model for arthritis and a tool for diagnosing a mammal's predisposition to joint disease.

Thus, the invention provides a rapid means for predicting and diagnosing ligament disease and damage in mammals, such as humans, dogs and horses. The invention also correlates the presence of particular bacterial species and the progression to joint ailments, particularly ligament damage.

In humans, diverse genera of bacteria can be found in approximately 10% of chronic arthritis patients. The presence of bacteria may not be clearly related to the specific clinical characteristics, which suggests that host genetic factors may be critically important to development of synovitis (Gerard et al. 2001). In this invention, it has been shown that the idiopathic arthropathy of chronic inflammatory knee arthritis and degenerative ACL rupture was significantly associated with the presence of mixtures of bacteria within the stifle joint (P<0.05); in healthy dogs the normal stifle joint is sterile. There was also a trend that dogs with two specific MHC class II DRB1 alleles (*0102 and *1502) were over-represented in dogs affected with the arthropathy (P=0.08). (Kennedy et al., Nine new dog ALA-DRB1 alleles identified by sequence-based typing, Immunogenetics 1998, 48:296-30; and, Kennedy et al. 2002). It has also been shown that dogs are considered a good sentinel for Borrelia infection. (Duncan et al. The dog as a sentinel for human infection: prevalence of Borrelia burgdorferi C6 antibodies in dogs from southeastern and mid-Atlantic states, Vector Borne Zoonotic Dis 2004, 4:221-229).

Using PCR methodology, the present invention demonstrates that bacterial DNA from several genera of bacteria is detected in the synovial membrane and synovial fluid of approximately 47% of dogs with cruciate rupture. This discovery will have profound implications for the diagnosis and medical treatment of this important canine condition that is currently unmet.

Synovial fluid lubricates and cushions the joints. A description synovial fluid is set forth in Remington, 21st Edition, at pages 351-352, which is incorporated herein by reference.

Various kits and methods of diagnosing degenerative joint disease are disclosed in U.S. Patent Application Publication No. US 2005/0074800, filed on Aug. 30, 2004, and published on Apr. 7, 2005, which is incorporated herein by reference.

Chronic joint inflammation is an important factor promoting progressive degradation of synovial joints. In inflammatory arthritides (such as rheumatoid arthritis, reactive arthritis, and chronic Lyme arthritis) dysregulation of antigen-specific T lymphocyte responses within the synovium is a key factor promoting persistent synovitis. In dogs, development of chronic inflammatory stifle arthritis and associated degenerative ACL rupture is a common naturally occurring condition. (Muir P et al., Vet Surg 2005, 34:239-46; and, Muir P et al., Vet Surg 2005, 34:482-90).

The synovium of affected dogs contains a mixture of inflammatory cells which include activated macrophages, B and T lymphocytes, and CD1c⁺ MHC class II⁺ dendritic cells. Histologically, in the normal canine stifle, very few synovial inflammatory cells are found. (Lemburg A K et al., Vet Immunol Immunopathol 2004, 97:231-40; and, Klocke N W et al., Am J Vet Res 2005, 66:493-9). Chronic synovitis is an important factor promoting progressive degradation of synovial joints over time. In the dog, persistent synovitis and development of inflammatory stifle arthritis is likely an important factor promoting degenerative ACL rupture, a common naturally occurring condition of most breeds of dog.

Taken together, these studies suggest that antigen presentation to T lymphocytes is a key pathway in the pathogenesis of persistent synovitis in this canine arthropathy. Although the triggering antigen or antigens involved in the joint immune responses in this arthropathy are unknown, other recent work suggests that environmental bacteria within joints are a possible trigger for the immune system. (Gerard H C et al., Arthritis Rheum 2001, 44:1689-97). In the present invention, the pathogenesis of synovitis in this canine inflammatory stifle arthritis model was determined. The phenotype of T lymphocytes within the synovium of affected dogs was determined. Quantified numbers of CD4⁺ helper T cells and CD8⁺ cytotoxic T cells within joint tissues and peripheral blood were also determined and large populations of activated synovial T lymphocytes (particularly CD4⁺ lymphocytes) were readily detectable within the stifle joints of affected dogs.

Degenerative rupture of the cranial cruciate ligament (CCL) is a common cause of lameness in the adult dog. (de Rooster H et al., Morphologic and functional features of the canine cruciate ligaments, Vet Surg 2006, 35:769-780). CCL rupture can occur in all breeds of dogs, although large and giant breed dogs are over-represented. (Hayashi K et al., Cranial cruciate ligament pathophysiology in dogs with cruciate disease: A review, J Am Anim Hosp Assoc 2004, 40:385-390). Both genders and all ages of dogs are affected. (Innes J F et al., Long-term outcome of surgery for dogs with cranial cruciate ligament deficiency, Vet Rec 2000, 147:325-328). Surgical treatment is usually indicated for dogs with unstable stifles. However, even with surgical treatment, weight-bearing remains abnormal and most dogs show reduction in jumping ability and tolerance to cold weather. (Innes et al. and Conzemius M G et al., Effect of surgical technique on limb function after surgery for rupture of the cranial cruciate ligament in dogs, J Am Vet Med Assoc 2005, 226:232-236).

Avulsion of the CCL can occur due to trauma, which is typically seen in young dogs. (Hayashi et al., 2004). In adult dogs, mid-substance rupture of the CCL during normal daily activity is typical. (de Rooster et al., 2006). Histologically, ruptured CCL contain decreased numbers of fibroblasts within the ligament, and chondroid metaplasia of fibroblasts from a fusiform to a spheroid phenotype (expansion of the volume of epiligamentous tissue) and significant disruption to the organized hierarchical structure of the extracellular matrix collagen. (Hayashi K et al., Histologic changes in ruptured canine cranial cruciate ligament, Vet Surg 2003, 32:269-277). Such findings suggest that CCL rupture is a progressive condition, whereby gradual deterioration in CCL mechanical properties eventually lead to mechanical overload, complete CCL rupture and development of stifle instability. (Heffron L E et al., Morphology, histology and functional anatomy of the canine cranial cruciate ligament, Vet Rec 1978, 102:280-283).

While the mechanism leading to gradual rupture of the CCL is not understood, CCL rupture is associated with development of stifle oligoarthritis. Oligoarthritis refers to an inflammatory arthritis affecting ≦4 joints. (Marzo-Ortega H et al., Early oligoarthritis, Rheum Dis Clin N Am 2005, 31:627-639). Both stifles are typically affected. Whilst it is clear that CCL transection contributes to the development of synovitis, development of synovitis and joint degradation may often precede CCL rupture. (Lipowitz A J et al., Synovial membrane changes after experimental transection of the cranial cruciate ligament in dogs, Am J Vet Res 1985, 46:1166-1170). Synovitis is known to degrade CCL structural properties and has been identified in dogs with stable stifles. (Goldberg V M et al., The influence of an experimental immune synovitis on the failure mode and strength of the rabbit anterior cruciate ligament, J Bone Joint Surg 1982, 64A:900-906).

Biomarkers of joint degradation are also elevated in the contralateral stifles of dogs with unilateral CCL rupture. (Steffey M A et al., The potential and limitations of cartilage-specific (V+C)(−) fibronectin and cartilage oligomeric matrix protein as osteoarthritis biomarkers in canine synovial fluid, Osteoarthr Cartil 2004, 12:818-825; and, de Bruin T et al., Evaluation of anticollagen type I antibody titers in synovial fluid of both stifle joints and the left shoulder joint of dogs with unilateral cranial cruciate disease, Am J Vet Res 2007, 68:283-289).

Synovitis typically involves the intima of the CCL epiligament and is characterized by the presence of B and T lymphocytes, IgG⁺, IgM⁺, and IgA⁺ plasma cells, macrophages and MHC II⁺ and CD1c⁺ dendritic cells. (Lemburg A K et al., Immunohistochemical characterization of inflammatory cell populations and adhesion molecule expression in synovial membranes from dogs with spontaneous cranial cruciate ligament rupture, Vet Immunol Immunopathol 2004, 97:231-240; Muir P et al., Evaluation of tartrate-resistant acid phosphatase and cathepsin K in ruptured canine cranial cruciate ligament in dogs, Am J Vet Res 2002, 63:1279-1284; and, Muir P et al., Localization of cathepsin K and tartrate-resistant acid phosphatase in synovium and cranial cruciate ligament in dogs with cruciate disease, Vet Surg 2005, 34:239-246).

Synovial inflammatory changes are similar to those found in canine rheumatoid arthritis. (Hewicker-Trautwein M et al., Immunocytochemical demonstration of lymphocyte subsets and MHC class II antigen expression in synovial membranes from dogs with rheumatoid arthritis and degenerative joint disease, Vet Immunol Immunopathol 1999, 67:341-357). Since the CCL is supplied with nutrients (not only from the overlying synovial vasculature but also from the synovial fluid of the stifle) development of stifle synovitis likely influences CCL metabolism. (Arnoczky S P et al, Microvasculature of the cruciate ligaments and its response to injury: An experimental study in dogs, J Bone Joint Surg 1979, 61:1221-1229; Whiteside L A et al., Nutrient pathways of the cruciate ligaments, J Bone Joint Surg 1980, 62A:1 176-1180; and, Kobayashi S et al., Microvascular system of the anterior cruciate ligament in the dog, J Orthop Res 2006, 24:1509).

Infiltration of inflammatory cells into CCL tissue and up-regulation of matrix-degrading proteases may be a key factor in the pathogenesis of degenerative CCL rupture. (Muir P et al., Collagen fragmentation in ruptured canine cranial cruciate ligament explants, Vet J 2006, 172:121-128). Co-localization of TRAP and cathepsin K has been shown in mononuclear cells within the CCL tissue. Infiltration of these mononuclear cells into the CCL and adjacent synovium is associated with increased fragmentation of collagen.

To further understand the pathogenesis of stifle oligoarthritis and associated degenerative CCL rupture in the dog, the instant invention involves determining the phenotype of lymphocytes within the synovial tissue of affected dogs and quantifying numbers of CD4+ helper T cells and CD8+ cytotoxic T cells within the joint tissues and peripheral blood. It is hypothesized that increased numbers of activated T lymphocytes, particularly CD4+ T lymphocytes, would be detectable within the synovial tissues of affected dogs.

### EXAMPLES

### Example 1

Blood and Joint Tissue Samples. Peripheral blood and specimens of synovium and ruptured ACL were collected from 23 dogs with inflammatory stifle arthritis/degenerative ACL rupture during surgical treatment. Peripheral blood samples were also collected from 4 healthy dogs with normal stifles. Procedures were conducted with the approval of the Animal Care Committee of the University of Wisconsin-Madison.

Preparation of PBMC for Flow Cytometry. PBMC were isolated using commercial cell separation tubes being BD Vacutainer™ CPT™, Becton Dickinson, Franklin Lakes, N.J.

After washing in phosphate buffered saline (PBS), erythrocytes were lysed. PBMC was then washed in RPMI with 10% fetal calf serum (RPMI/FCS). After determining the concentration of cells, cell counts were adjusted to ≦1×10⁷/ml using FACS buffer. PBMC were then blocked and stained with fluorochrome-labeled anti-canine monoclonal antibodies directed against CD3, CD4, CD8, and CD44 epitopes, Serotec, Raleigh, N.C.) and fixed in 2% paraformaldehyde in PBS pH 7.4 for FACS analysis. CD3 staining was used to confirm the identity of CD4⁺ and CD8⁺ cells as T lymphocytes.

Preparation of Synovial Mononuclear Cells for Flow Cytometry. Joint tissues consisting of synovial membrane and ruptured ACL were placed in a Petri dish and washed with PBS. RPMI/FCS was then added. The tissues were minced using a scalpel blade. Minced tissues were suspended in 20 ml of RPMI/FCS with 4 mg/ml of collagenase B. The cell suspension was incubated at 37° C. for 1.5 to 2.5 hours. The cell suspension was agitated every 20 minutes during this incubation. Mononuclear cells were then isolated using Percoll density gradient centrifugation. Mononuclear cells were then washed in RPMI/FCS, and erythrocytes were lysed. Joint mononuclear cells were then stained with fluorochrome labeled antibodies, as described above.

FACS Analysis. The percentage of CD4⁺ and CD8⁺ T lymphocytes was determined using a FACS Calibur flow cytometer (BD Biosciences, San Jose, Calif.). The flow cytometry data were analyzed using CellQuest software (BD Biosciences).

Data Analysis. Differences in numbers of CD4⁺ and CD8⁺ T lymphocytes in peripheral blood and stifle synovial tissue were analyzed using the Student's t test for paired and unpaired data. Where data was not parametrically distributed, the Mann Whitney U test and the Wilcoxon matched-pairs tests were used. Results were considered significant at P<0.05.

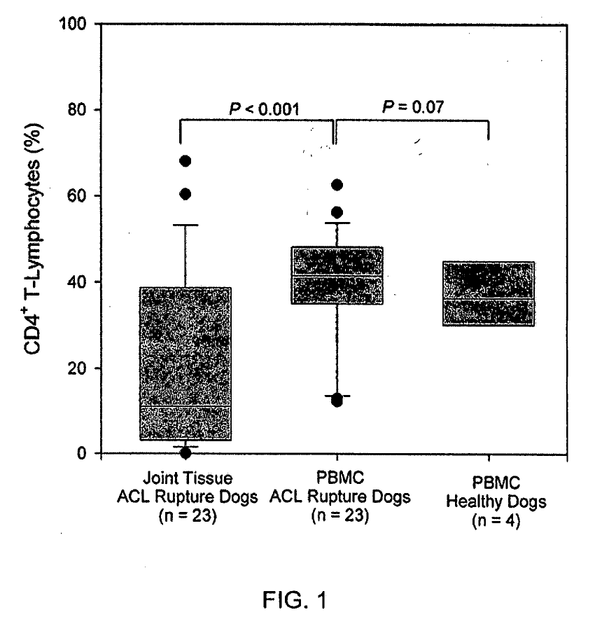

Results. Large numbers of CD4⁺ and CD8⁺ T lymphocytes were detected in the stifle joints of affected dogs (CD4—0.25×10⁶/ml±0.37×10⁶/ml and CD8—0.11×10⁶/ml±0.14×10⁶/ml, mean±standard deviation respectively. (See FIGS. 1-3). In the peripheral blood of ACL arthropathy dogs, numbers of CD4⁺ and CD8⁺ T-lymphocytes were 1.10×10⁶/ml±0.69×10⁶/ml and 0.52×10⁶/ml±0.51×10⁶/ml respectively. In the peripheral blood of healthy dogs, numbers of CD4⁺ and CD8⁺ T-lymphocytes were 0.44×10⁶/ml±0.10×10⁶/ml and 0.19×10⁶/ml±0.02×10⁶/ml respectively.

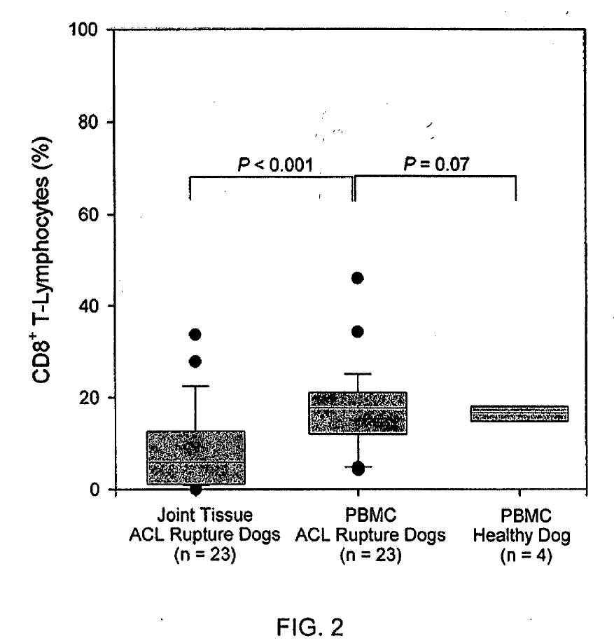

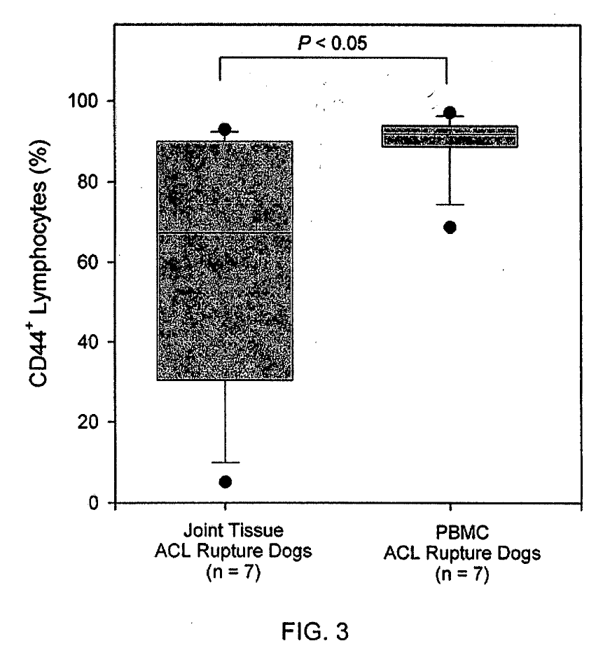

Numbers of CD4⁺ and CD8⁺ T-lymphocytes were higher in the peripheral blood of ACL arthropathy dogs when compared with healthy dogs. (P=0.07)(See FIGS. 1 and 2). As shown in FIG. 3, expression of CD44 (i.e., CD44⁺ lymphocytes) was also found in 89% of PBMC and 58% of joint mononuclear cells in dogs with chronic stifle synovitis/degenerative ACL rupture. Large numbers of CD4⁺ and CD8⁺ T lymphocytes were detected in the stifle joints of affected dogs. A large proportion of the mononuclear cells within the stifle joints also expressed the activation marker CD44. (See FIG. 3). Boxes represent median and the 25th and 75th percentiles. Whiskers represent 10th and 90th percentiles. Outliers are also plotted dogs. Numbers of T lymphocytes infiltrating the stifle synovium and ruptured ACL remnants were variable between individual dogs.

Although it is generally accepted that dysregulation of local immune responses within joints is a key factor in the development of persistent synovitis, the immune mechanisms involved are poorly understood and likely complex. Data from the present invention shows that large numbers of activated T lymphocytes are present within joint tissues. This suggests that antigen-specific immune responses are likely an important factor in the pathogenesis of chronic inflammatory arthritis and progressive joint degradation. In addition, mixtures of bacteria are often detectable within the synovium and synovial fluid of affected joints in this invention. It is further hypothesized that bacterial antigens may be a key factor triggering dysregulation of joint innate and antigen-specific immune responses in this invention. The presence of mixtures of bacteria or bacterial DNA within joints may increase the likelihood that persistent synovitis develops over time. (Gerard H C et al., 2001).

Synovial pathology in this canine condition is similar to human inflammatory arthritides, such as RA. In RA, dysregulation of antigen-specific immune responses is a key feature promoting synovitis. Inflammatory changes in ruptured human ACL are less extensive. (Barrett J G et al., Am J Vet Res 2005, 66:2073-2080).

The pathogenesis of joint inflammation in this canine stifle arthritis model is determined by the present invention. The present invention demonstrates the pattern of immune response gene expression in dogs with inflammatory stifle arthritis/degenerative ACL rupture. It is hypothesized that immune-response gene expression would be up-regulated in dogs with inflammatory stifle arthritis.

Blood and Joint Tissue Samples. Peripheral blood and synovial fluid samples were collected from 32 dogs with inflammatory stifle arthritis/ACL rupture during surgery, 8 healthy dogs with normal stifles and intact ACL, and 9 dogs without ACL rupture and with degenerative osteoarthritis of a large synovial joint. Procedures were conducted with the approval of the Animal Care Committee of the University of Wisconsin-Madison. PBMC were isolated using commercial cell separation tubes (BD Vacutainer™ CPT™, Becton Dickinson, Franklin Lakes, N.J.). Synovial fluid cells were isolated by centrifugation.

Quantitative RT-PCR. mRNA expression was quantified in PBMC and synovial fluid cells. Total RNA was isolated using standard RNAzol B methodology. Quantitative RT-PCR (qRT-PCR) was performed using a BioRad real-time thermocycler and commercially available SYBR green kits. Oligonucleotide primers for the genes of interest were designed for antigen-specific immune response genes (i.e., cathepsin S, TRAP, and invariant chain) and matrix turnover genes (i.e., cathepsin K, MMP-9, cathepsin S). PCR reactions were performed in duplicate.

Data Analysis. For each sample, the threshold cycle (Cₜ values) obtained from the exponential region of the PCR amplification plot from the duplicate trials were averaged together. Relative gene expression for each of the genes-of-interest was then calculated using the −ΔΔCt method. (Livak K J et al., Methods 2001, 25:402-8). PBMC gene expression was used as an internal control, and the 18S rRNA gene was used as the housekeeping gene. Relative mRNA expression was calculated as 2^{-(average ΔΔCt)}. After log-transformation, a Student's t test with a hypothesized mean equal to zero was used to determine whether synovial fluid gene expression was significantly different from PBMC (internal control). One-way ANOVA and a post-hoc t test were used to determine differences between groups. Differences were considered significant at P<0.05.

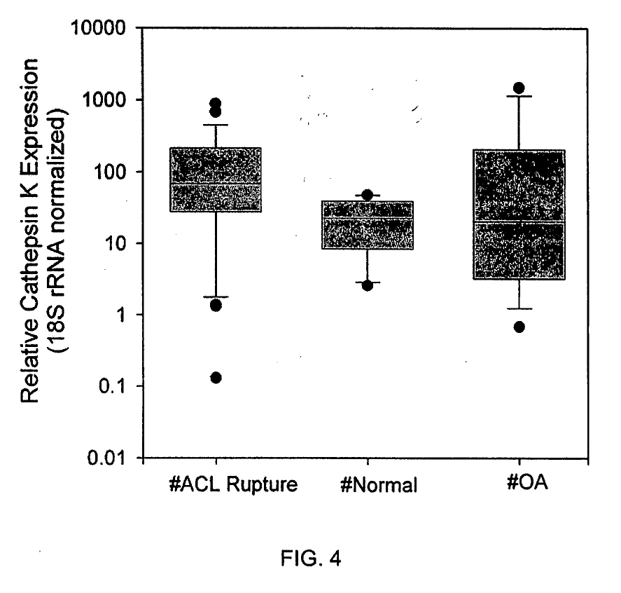

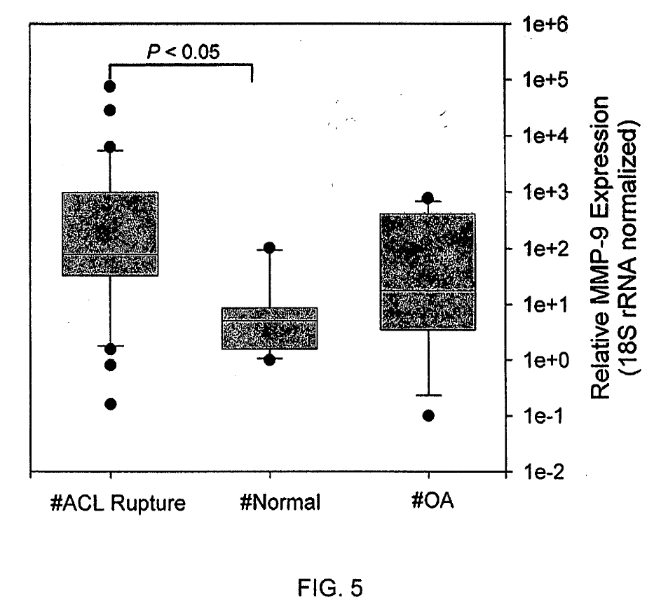

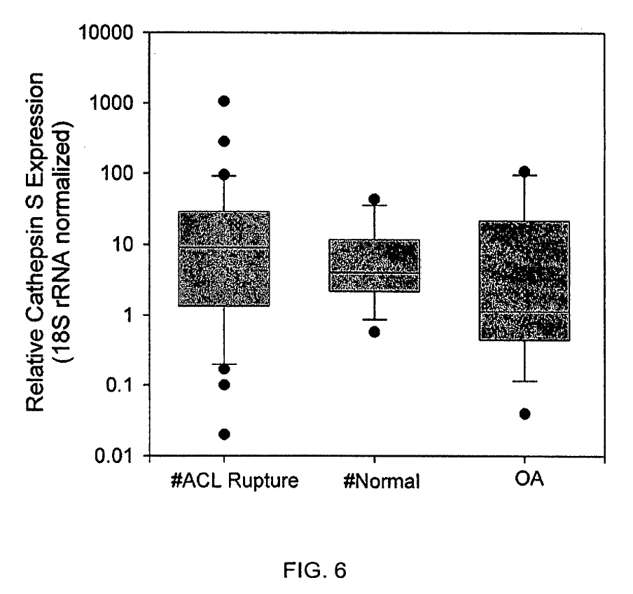

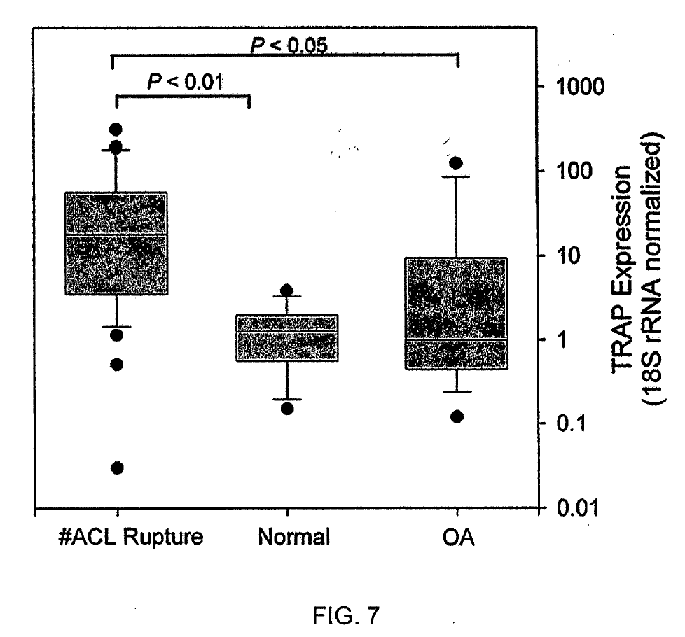

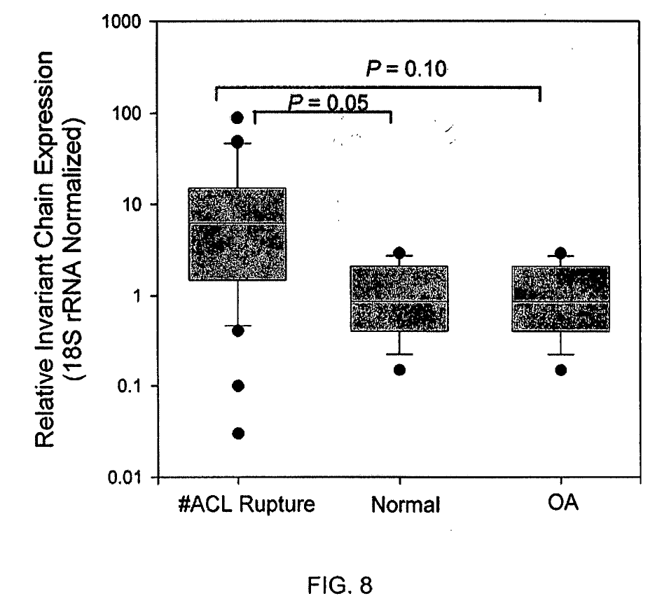

Results are summarized in FIGS. 4-8. Relative expression of the MMP-9 (196-fold), TRAP (35-fold), and invariant chain (14-fold) genes was significantly increased in the stifle synovial fluid of dogs with ACL rupture when compared with the stifles of normal dogs (P≦0.05). In contrast, relative expression of all of the genes-of-interest in synovial fluid from joints affected with osteoarthritis was not significantly different when compared with the stifles of normal dogs.

In the ACL rupture dogs, expression of cathepsin K (167-fold), MMP-9 (4053-fold), cathepsin S (60-fold), TRAP (51-fold), and invariant chain (15-fold) was significantly increased in stifle synovial fluid when compared with the internal PBMC control. In normal dogs, only cathepsin S (10-fold), cathepsin K (24-fold), and MMP-9 (21-fold) were significantly increased in stifle synovial fluid when compared with the internal PBMC control (P<0.05). In dogs with osteoarthritis, only expression of cathepsin K (254-fold) and MMP-9 (208-fold) were increased in synovial fluid when compared with the internal control (P<0.05).

It is generally accepted that dysregulation of local immune responses within joints is a key factor in the development of persistent synovitis and progressive joint degradation. However, the immune mechanisms involved are poorly understood and likely complex. In normal dogs and dogs with osteoarthritis, matrix turnover genes were primarily up-regulated in joint tissues when compared with PBMC internal controls. Both antigen-specific immune response genes and matrix turnover genes were up-regulated in dogs with inflammatory stifle arthritis/degenerative ACL rupture.

In particular, expression of TRAP and invariant chain was increased in dogs with the ACL rupture arthropathy when compared with normal dogs. Levels of expression of these genes were also higher when compared with dogs with osteoarthritis (P<0.05 and P=0.1, respectively). These data demonstrate that levels of expression of TRAP and invariant chain genes are useful biomarkers for inflammatory arthritis in this canine model. Although the key triggering antigens are unknown, expression of TRAP in macrophages is thought to have a key role in immune clearance of bacteria. (Bune A J et al., Immunology 2001, 102:103-113).

As shown in FIGS. 4-8, the present invention also demonstrates that antigen-specific immune responses are, at least in part, an important factor in the pathogenesis of synovitis. Relative gene expression in the stifle synovial fluid was normalized to PBMC as an internal control. 18S rRNA was used as the housekeeping gene. # indicates a significant difference from the internal PBMC controls (P<0.05). Boxes represent median and the 25th and 75th percentiles. Whiskers represent 10th and 90th percentiles. Outliers are also plotted. Data were summarized from 32 ACL rupture dogs, 8 normal dogs, and 9 dogs with osteoarthritis (OA).

Bacteria are often found in joints affected with inflammatory arthritis. Mixtures of bacteria within joints have been implicated as a causative factor in the pathogenesis of arthritis. Polymicrobial populations of bacteria within synovial joints likely increase the risk that bacteria trigger immune system dysregulation and development of persistent synovitis and associated joint degeneration.

Inflammatory stifle arthritis/degenerative ACL rupture is a common naturally occurring condition of the dog. In this disease, the stifle synovium is infiltrated with a mixed population of inflammatory cells, which include B & T lymphocytes, macrophages, and dendritic cells. (Lemburg et al., 2004). Expression of immune response genes is up-regulated in affected joints. These findings suggest that antigen-specific immune responses are likely important in the pathogenesis of synovitis in this canine arthropathy. It is hypothesized that bacterial DNA is detectable in dogs with inflammatory arthritis/degenerative ACL rupture, but not dogs with normal stifles or experimentally-induced ACL rupture.

Dogs. Experiment #1. Synovial membrane specimens were collected during surgical treatment of 43 dogs for ACL rupture and associated stifle instability. In addition, specimens of synovium were collected from 12 normal dogs with intact ACL, and 16 Pond-Nuki dogs in which unilateral ACL rupture was induced experimentally in a normal stifle for a period of 19 weeks.

### Example 2

Both synovial fluid and synovial membrane specimens were collected at surgery from a further 51 dogs with inflammatory stifle arthritis/degenerative ACL rupture. Dogs with PCR-positive joints were treated with doxycycline at 5 mg/kg bid orally for 10 weeks. Follow-up synovial fluid specimens were then collected aseptically by percutaneous needle aspiration.

Specimen collection and PCR for Bacterial DNA. Experiment #1. Synovial membrane specimens were collected aseptically during surgery. For the initial experiment, after extraction of DNA, a non-nested broad-range panbacterial PCR method was used for detection of DNA from the bacterial 16S rRNA gene. The PCR system used consensus primers for a highly conserved region of the gene. (Gerard H C et al., 2001).

All PCR reactions were performed in a laminar flow hood and extensive precautions were taken to prevent contamination of the PCR system. PCR products were examined under UV light after electrophoresis on a 1.5% agarose gel and staining with ethidium bromide. PCR-positive specimens were cloned. At least 4 clones were prepared from each synovial membrane specimen. Bacterial species were identified by a BLAST search of GenBank. In addition species-specific non-nested PCR methods were used to detect DNA from the Borrelia burgdorferi outer surface protein A (OspA) and 66-kDa protein (p66) genes. PCR-positive specimens were sequenced to confirm specificity.

Synovial fluid cells were centrifuged to isolate the cell pellet. The panbacterial 16S rRNA and OspA PCR methods were used to test both synovial membrane and synovial fluid cell specimens for bacterial DNA sequences before and after doxycycline treatment. PCR products were examined in UV light after electrophoresis on a 1.5% agarose gel and staining with ethidium bromide.

Non-nested PCR analysis of both synovial membrane and synovial fluid in dogs with stifle arthritis/degenerative ACL rupture was performed, and the proportion of PCR-positive dogs increased to 47%. Non-nested PCR methods for panbacterial 16S rRNA and the Borrelia burgdorferi OspA gene were performed on the both the synovial membrane and synovial fluid in 51 dogs treated surgically for ACL rupture. OspA DNA sequences were detectable as part of a polymicrobial population of joint bacteria (4 of 6 dogs). Established molecular microbiological guidelines for microbial causation (Fredericks D N et al., Sequence-based identification of microbial pathogens: a reconsideration of Koch's postulates, Clin Microbiol Rev 1996, 9:18-33) supports the theory that there exists a causative relationship between polymicrobial populations of joint bacteria and the development of inflammatory stifle arthritis, joint degeneration, and ACL rupture in dogs, which explains the increase in the proportion of affected dogs testing PCR-positive.

Statistics. The Fisher's Exact test was used to compare the proportion of dogs in each group with bacteria-positive stifle tissues. Values of P were one-sided. Values of P<0.05 were considered significant.

Dogs with bacterial DNA-positive stifles. In Experiment #1, the presence of bacterial DNA in the stifle synovium was significantly associated with the inflammatory stifle arthritis/degenerative ACL rupture arthropathy. Synovial membrane specimens from 16 of 43 dogs with the ACL rupture arthropathy (37%) were PCR-positive. A 16S rRNA PCR product was found in 14 dogs. Of these dogs, stifle synovium from four dogs was also positive for Borrelia burgdofferi DNA. The remaining two dogs were positive for OspA only. None of the 12 specimens collected from dogs with normal stifles and intact ACL were PCR-positive. One of the 16 synovial specimens collected from Pond-Nuki dogs was positive for OspA.

Response to doxycycline therapy. In Experiment #2, where PCR of both synovium and synovial fluid was performed in dogs with the ACL rupture arthropathy, 47% of stifles were PCR-positive. Both 16S rRNA and OspA DNA were detectable with similar frequencies in synovium (16S rRNA—33%; OspA—5.9%) and synovial fluid (16S rRNA—37%; OspA—5.9%). Again, OspA DNA sequences were usually detectable together with 16S rRNA DNA sequences (5 of 7 dogs).

Stifle joints of dogs affected with arthritis/degenerative ACL rupture that are 16S rRNA PCR-positive for bacteria became PCR-negative after treatment with oral doxycycline administered 5 mg/kg bid for 10 weeks (n=6 dogs). One dog that was initially OspA PCR-positive remained PCR-positive after the doxycycline treatment (n=1 dog). PCR methods were used to analyze the joint tissues in dogs with inflammatory stifle arthritis and degenerative ACL rupture before and after doxycycline treatment.

For 16S rRNA PCR-positive joints, doxycycline had a significant positive treatment effect (P<0.05). 6 dogs were in the trial: 6 dogs were PCR-positive for panbacterial 16S rRNA gene and 1 dog was also PCR-positive for the OspA gene. Follow-up synovial fluid specimens were collected after doxycycline treatment: All 6 dogs were PCR-negative for the panbacterial 16SrRNA gene. After treatment, the dog which was PCR-positive for the OspA gene remained OspA positive; the other 5 dogs remained PCR-negative for the OspA gene. Synovial fluid was tested at the time of surgical treatment for ACL rupture with tibial plateau leveling osteotomy. Synovial fluid was aspirated percutaneously at 10 weeks after surgery at the time of recheck radiography for tibial osteotomy healing. The data demonstrate that doxycycline therapy significantly reduces joint bacteria, P<0.05, Fisher Exact test was used.

That data suggest that the mixtures of bacteria present in stifle joints of at least 50% of affected dogs are metabolically viable. The data also shows that antibiotics, such as doxycycline, effectively treat polymicrobial bacteria-associated chronic arthritis. The presence of viable bacteria may not, however, be essential for inducing joint inflammation. (Deng G-M et al., The features of arthritis induced by CpG motifs in bacterial DNA, Arthritis Rheum 2000, 43:356-64).

Bacterial species identified in dogs with degenerative ACL rupture arthropathy. DNA from a wide range of bacterial species was identified in the dogs with the degenerative ACL rupture arthropathy. By sequencing of multiple clones, mixtures of bacteria were identified in 13 of 14 dogs. In addition to Borrelia burgdorferi (14% of dogs), other organisms commonly identified were uncultured Eubacterium species (21%), Stenotrophomonas maltophilia (14%), Rhizobium radiobacter (9%), and Ralstonia solanacearum (7%) uncultured beta Proteobacterium (7%), Achromobacter xylosoxidans (5%), uncultured Oxalobacterium (5%), Corynebacterium glutamicum (5%), Pantoea agglomerans (2%), Rhizobium galegae (2%), Gordonia terrae (2%), Acinetobacter calcoaceticus (2%), Pseudomonas putida (2%), uncultured Gemmatiomonadates bacterium (2%), uncultured Burkholderia (2%).

DNA from a wide variety of bacterial species can often be found in the synovium of arthritic joints from human patients. (Gerard H C et al., 2001). It is generally accepted that dysregulation of local immune responses within joints is a key factor in the development of persistent synovitis and progressive degradation of synovial joints. However, such immune mechanisms involved are poorly understood and likely complex.

The present invention establishes a significant association between the presence of bacterial DNA within synovium and the canine degenerative ACL rupture arthropathy. Analysis of both synovium and synovial fluid shows that the proportion of affected dogs with PCR-positive stifles was 47%, which is a higher proportion compared with equivalent human studies. In healthy dogs with normal stifle joints, all joints were PCR-negative suggesting that the normal canine stifle is sterile. In the present invention, data from healthy dogs with induced ACL rupture suggests that translocation of mixtures of bacterial to the stifle is not simply a consequence of joint instability.

Borrelia burgdorferi was the only recognized joint pathogen identified. Borrelia burgdorferi was most often found together with mixtures of environmental bacteria suggesting that mixtures of bacteria are important for development of synovitis. Chronic Lyme arthritis develops preferentially in humans with specific MHC class II genotypes. The MHC class II gene complex of dogs models that of humans. Therefore, it is theorized that a similar genetic susceptibility exists in the dog, particularly dogs with the DRB1 alleles *0102 and *1502.

OspA contamination is a possible explanation for identification of Borrelia burgdorferi in one Pond-Nuki dog and two degenerative ACL rupture arthropathy dogs that were panbacterial PCR negative. Elimination of the 16S rRNA PCR product from joints after treatment with the bacteriostatic drug doxycycline suggests that mixtures of environmental bacteria within joints are viable. It is further hypothesized that the presence of mixtures of environmental bacteria and bacterial DNA within synovial joints is a key factor triggering persistent synovitis and progressive ACL rupture. It is known that chronic synovitis degrades ACL structural properties. (Goldberg V M et al., J Bone Joint Surg 1982, 64A:900-6).

DLA-DRB1*0102 and DLA-DRB1*1502 alleles are over-represented in dogs with inflammatory stifle arthritis/ACL rupture and stifle synovium that is PCR-positive for bacterial DNA. An oligonucleotide DRB1 tailed primer set was used for sequence-based typing of DLA-DRB1. The primer information is set forth below in Table 1.

TABLE 1

| | Primer | | Amplicon |
|---|---|---|---|
| DNA Targets | Type | Oligonucleotides (5′ to 3′) | Size (bp) |
| DRB1 M13 tailed | Forward | TGTAAAACGACGGCCAGTGATCCCCCCG | 303 |
| primers | | TCCCCACAG | |
| | Reverse | CAGGAAACAGCTATGACCCGCCCGCTGC | |
| | | GCTCA | |
| DQA M13 tailed | Forward | TGTAAAACGACGGCCAGTTAAGGTTCTT | 345 |
| primers | | TTCTCCCTCT | |
| | Reverse | CAGGAAACAGCTATGACCGGACAGATTC | |
| | | AGTGAAGAGA | |
| DQB | Forward | CTCACTGGCCCGGCTGTCTC | 300 |
| | Reverse | CACCTCGCCGCTGCAACGTG | |
| M13 Cycle | Forward | TGTAAAACGACGGCCAGT | 450 |
| Sequencing | Reverse | CAGGAAACAGCTATGACC | |
| Primers | | | |

The primers are intronic and locus specific. PCR and direct sequencing were also used. DLA-DRB1 alleles were compared in a group of 15 dogs with inflammatory stifle arthritis/degenerative ACL rupture and a group of 15 breed-matched controls. Breed-matched controls were used to increase the statistical power of the comparison. Variation in MHC class II alleles was greater between breeds than within a breed due to inbreeding.

As shown in Table 2 below, two DRB1 alleles are over-represented in affected dogs: DRB1*0102 and DRB1*1502. Table 2 shows DLA-DRB1 genotype and phenotype frequencies in dogs with inflammatory stifle arthritis/degenerative ACL rupture and breed-matched control dogs. Neither of those alleles contain RA shared epitope sequences. It was also found that the incidence of DRB1 homozygosity was 0.47 in dogs with inflammatory arthritis/degenerative ACL rupture and 0.25 in breed-matched controls. (P=0.25). Thus, the mechanisms by which bacteria trigger synovitis in the present model involves the MHC complex. The mechanisms are also antigen-specific.

TABLE 2

| | Genotype Comparison | | | Phenotype Comparison | | |
|---|---|---|---|---|---|---|
| | ACL | | | ACL | | |
| | rupture | Controls | | rupture | Controls | |
| | (n = 15 | (n = 15 | P | (n = 15 | (n = 15 | P |
| Allele | dogs) | dogs) | value | dogs) | dogs) | value |
| DRB1*0101 | 0.03 | 0.1 | 0.3 | 0.07 | 0.2 | 0.28 |
| DRB1*0102 | 0.1 | 0.00 | 0.08 | 0.13 | 0.00 | 0.14 |
| ¹DRB1*0201 | 0.03 | 0.03 | 1.00 | 0.07 | 0.07 | 1.00 |
| DRB1*0401 | 0.00 | 0.07 | 0.15 | 0.00 | 0.13 | 0.14 |
| DRB1*0601 | 0.27 | 0.17 | 0.35 | 0.33 | 0.27 | 0.69 |
| ¹DRB1*0802 | 0.03 | 0.07 | 0.55 | 0.07 | 0.13 | 0.54 |
| DRB1*0901 | 0.07 | 0.03 | 0.55 | 0.13 | 0.07 | 0.54 |
| ¹DRB1*1101 | 0.00 | 0.03 | 0.31 | 0.00 | 0.07 | 0.30 |
| DRB1*1201 | 0.13 | 0.17 | 0.72 | 0.20 | 0.33 | 0.4 |
| DRB1*1501 | 0.13 | 0.17 | 0.72 | 0.27 | 0.2 | 0.67 |
| DRB1*1502 | 0.1 | 0.00 | 0.08 | 0.13 | 0.00 | 0.14 |
| DRB1*1503 | 0.03 | 0.03 | 1.00 | 0.07 | 0.07 | 1.00 |
| ²DRB1*018v | 0.00 | 0.07 | 0.15 | 0.00 | 0.07 | 0.30 |
| DRB1*2001 | 0.03 | 0.03 | 1.00 | 0.07 | 0.07 | 1.00 |
| DRB1*2301 | 0.03 | 0.00 | 0.31 | 0.07 | 0.00 | 0.3 |
| DRB1*2601 | 0.00 | 0.03 | 0.31 | 0.00 | 0.07 | 0.30 |

¹DRB1 alleles containing RA shared epitope sequences in the third hypervariable regions.
²Novel allele not currently recorded in the EBI database.

### Example 3

Blood and Joint Tissue Samples. Peripheral blood, synovium, and the ruptured ends of the CCL, when available, were collected from 23 dogs with stifle oligoarthritis and associated degenerative CCL rupture during surgical treatment. Age, weight, and gender were recorded for each dog. Peripheral blood and synovial tissues were also collected from 6 normal beagles with healthy stifle joints as a baseline for comparison with the dogs with oligoarthritis. The Beagles were all female; body weight and age were 10.5±1.7 kg and 3.3±3.0 years. Peripheral blood samples were also collected from five young healthy dogs of other breeds (Airedale Terrier, Pitbull Terrier, Toy Poodle, Gordon Setter and Boxer). The age and weight of these dogs were 21.5±9.8 kg and 1.5±1.4 years.

Preparation of Peripheral Blood Mononuclear Cells (PBMC) for Flow Cytometry. PBMC were isolated using commercial cell separation tubes (BD Vacutainer™ CPT™, Becton Dickinson, Franklin Lakes, N.J.). After washing in RPMI media (Sterile-filtered RPMI-1640 with Penicillin, Streptamycin, L-Glutamine and 24 mM Hepes) and 10% Fetal Calf Serum (RPMI/FCS), erythrocytes were lysed with 1 ml of 0.83% ammonium chloride. PBMC were then washed with RPMI/FCS. Cells were counted and adjusted to ≦1×107 cells/ml using FACS buffer for flow cytometry. PBMC were blocked on ice using FACS buffer and were then stained with fluorochrome-labeled monoclonal antibodies for anti-canine CD3, CD4, CD8, and CD44 (Serotec, Raleigh, N.C.). CD3 was used as a T lymphocyte marker to confirm the identity of CD4+ and CD8+ cells. CD44 was used as a lymphocyte activation marker. Cells were fixed in 2% paraformaldehyde in PBS pH 7.4 and stored protected from light at 2-8° C. prior to FACS analysis.

Preparation of Synovial Mononuclear Cells for Flow Cytometry. Synovial membrane and ruptured CCL tissues were placed in a petri dish and washed with PBS. Tissues were then suspended in RPMI/FCS and gently disrupted using a scalpel blade and then a syringe plunger. Mononuclear cells were then isolated using a 100 μm cell strainer and suspended in RPMI/FCS. Mononuclear cells were isolated using Percoll density gradient centrifugation. Mononuclear cells were then washed in RPMI/FCS and erythrocytes were lysed with ammonium chloride. Joint mononuclear cells were then blocked and stained with fluorochrome labeled antibodies, as described above.

FACS Analysis. The percentage of CD4+ and CD8+ T lymphocytes was determined using a FACS Calibur flow cytometer (BD Biosciences, San Jose, Calif.). Flow cytometry data were analyzed using CellQuest software (BD Biosciences) and FlowJo software (TreeStar, Inc., Ashland, Oreg.).

Data Analysis. The Kolmogorov-Smirnoff test was used to determine whether sample data were normally distributed. Differences in numbers of CD4+ and CD8+ T lymphocytes in peripheral blood and stifle synovial tissues were analyzed using the Student's t test for paired and unpaired data. For data that was not parametrically distributed, the Mann Whitney U test and the Wilcoxon matched-pairs tests were used. Results were considered significant at P<0.05.

Results. The breeds of the 23 dogs with oligoarthritis and associated degenerative CCL rupture were Labrador retriever (n=5), Golden Retriever (n=4), Rottweiler (n=2), Rottweiler cross (n=2), Beagle (n=2), Labrador cross (n=1), Newfoundland (n=1), Pit Bull Terrier (n=1), Pit Bull Terrier cross (n=1), Gordon Setter (n=1), Springer Spaniel (n=1), Weimeraner (n=1), and mix breed (n=1). Body weight and age were 37.9±13.6 kg and 5.1±2.8 years, respectively. Eight dogs were ovariohysterectomized females, 11 dogs were castrated males, 3 dogs were female, and 1 dog was male. Median duration of lameness was 12 weeks (range 1-88 weeks). Ten dogs had a clinical history of bilateral CCL rupture. All of the dogs with CCL rupture had palpable instability of the stifle on physical examination, indicating complete cruciate rupture.

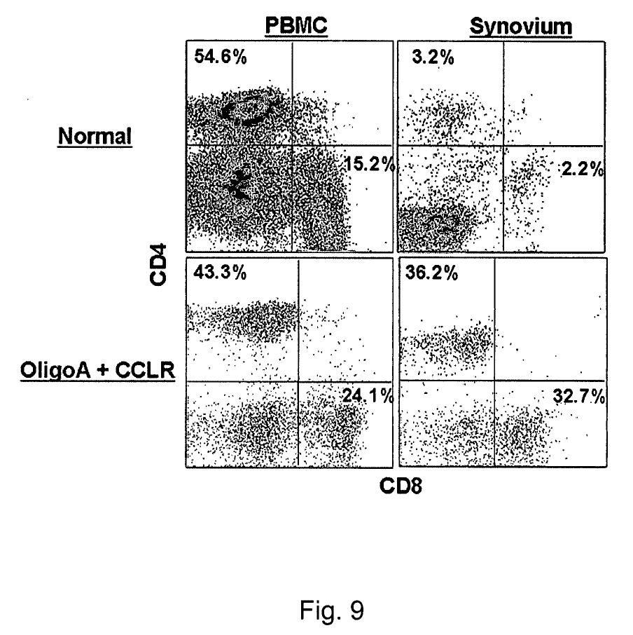

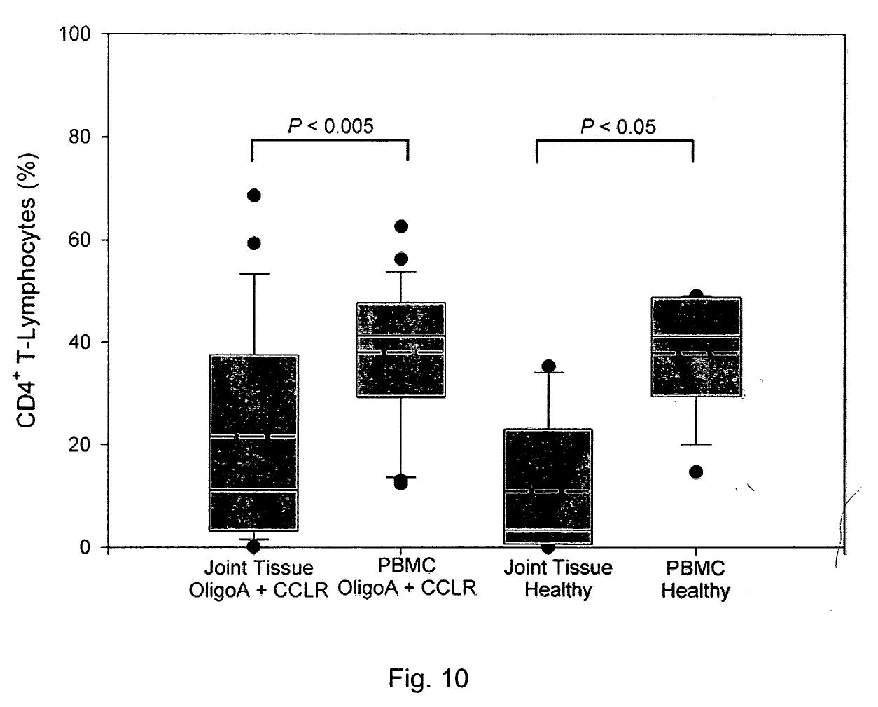

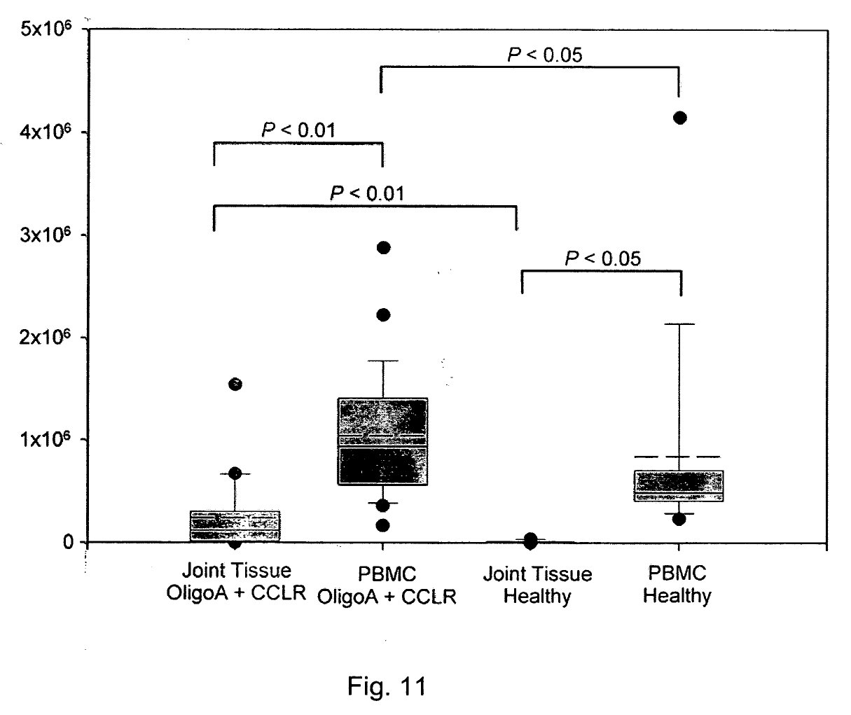

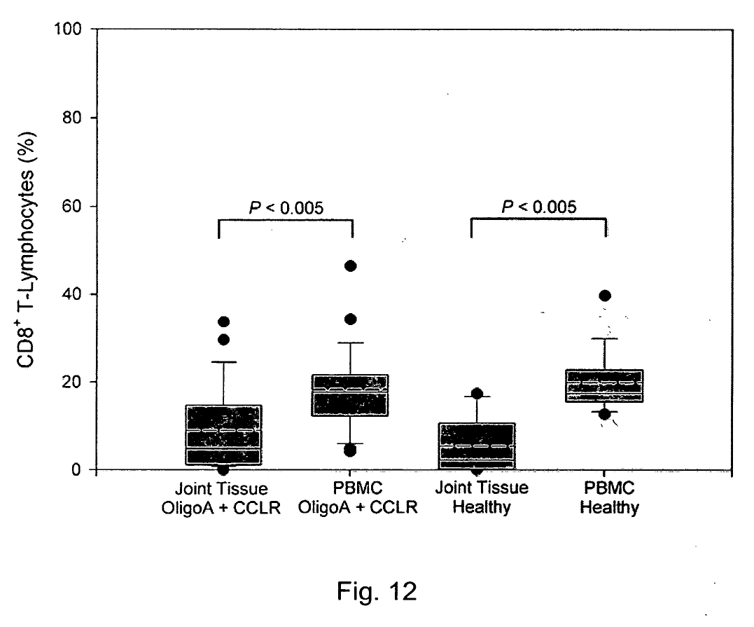

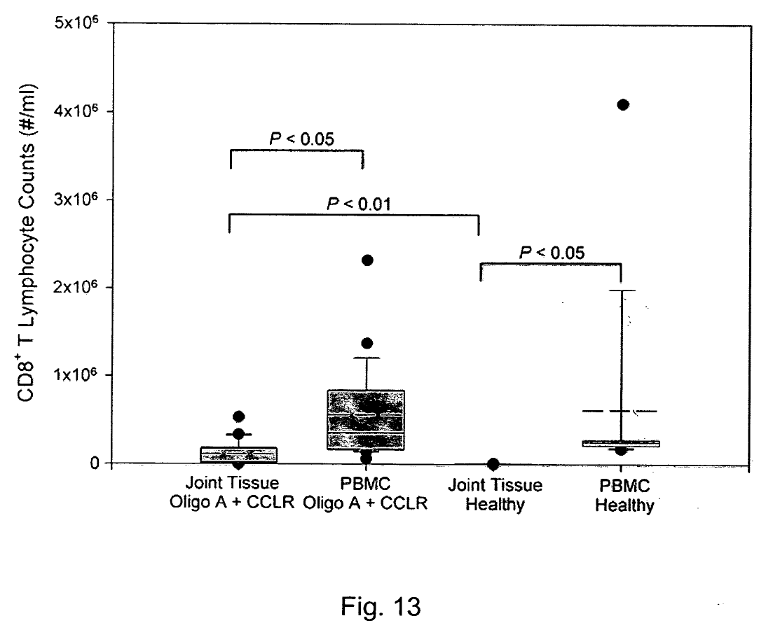

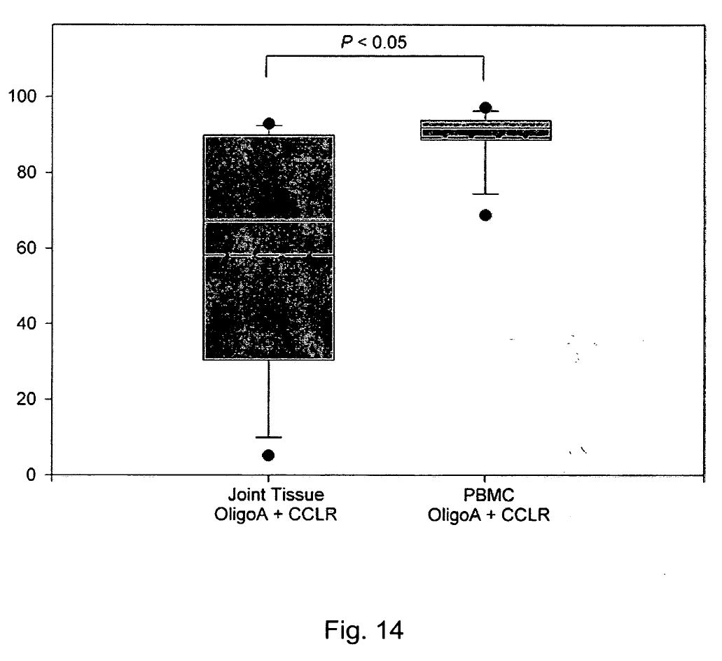

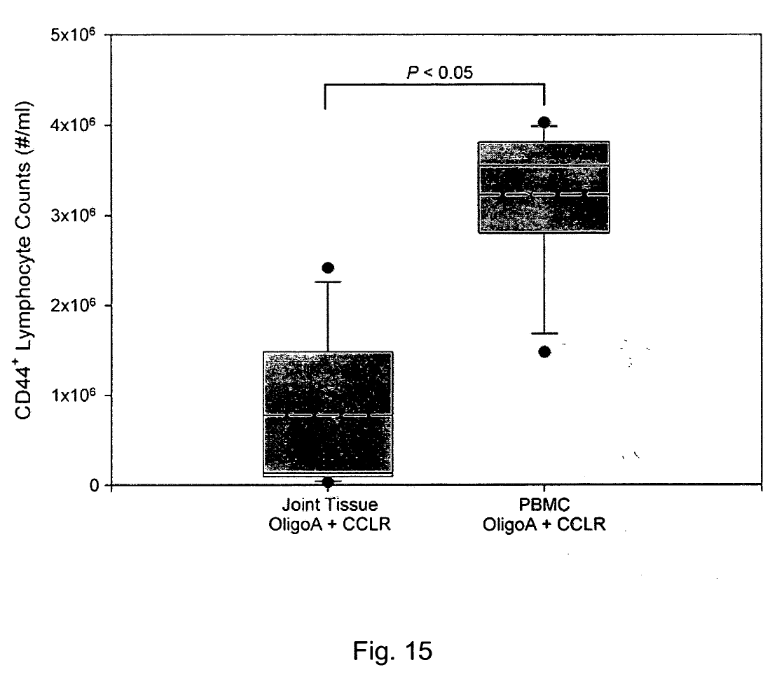

The population of CD4+ and CD8+ T lymphocytes within the stifle synovium of healthy dogs was quite small; numbers of T lymphocytes within the stifle synovium were significantly increased in dogs with stifle oligoarthritis as shown in FIGS. 9-15. (P<0.005). Percentages of CD4+ and CD8+ T lymphocytes in joint tissues and peripheral blood were similar in both healthy dogs and dogs with stifle oligoarthritis and associated degenertive CCL rupture. The proportion of T lymphocytes within the peripheral blood was higher than the stifle synovium in both groups of dogs as shown in FIGS. 10-15. Numbers of CD4+ T lymphocytes were also increased in the peripheral blood of dogs with oligoarthritis, when compared with healthy dogs as shown in FIGS. 10-15. (P=0.05). In all dogs, numbers of CD4+ and CD8+ T lymphocytes were increased in the peripheral blood, when compared with the stifle synovium (P<0.05). Expression of the activation marker CD44 was also found in 89% of PBMC and 58% of joint mononuclear cells in dogs with oligoarthritis. (See FIGS. 10-15). Numbers of T lymphocytes infiltrating the stifle synovium and ruptured CCL remnants were variable between individual dogs.

The data in Table 3 shows the numbers of CD4⁺ and CD8⁺ T-lymphocytes within stifle synovium and peripheral blood of dogs with CCL rupture arthropathy, whereby CD4⁺ are helper T-lymphocytes and CD8⁺ are cytotoxic T-lymphocytes.

TABLE 3

| | Stifle Tissue | | PMBC | |
|---|---|---|---|---|
| | CD4⁺ | CD8⁺ | CD4⁺ | CD8⁺ |
| CCL | 0.24 ± 0.36** | 0.11 ± 0.15** | 1.04 ± 0.65* | 0.55 ± 0.52 |
| Rupture | | | | |
| Healthy | 0.01 ± 0.01 | 0.00 ± 0.00 | 0.84 ± 1.11 | 0.62 ± 1.16 |

*P < 0.05
**P < 0.01 when compared with healthy dogs

### Example 4

Collection of Specimens of Synovial Fluid and Synovial Membrane. Both synovial fluid and synovial membrane specimens were collected aseptically from 130 dogs affected with inflammatory stifle arthritis/degenerative CCL rupture at the time of surgical treatment over a one year duration. Specimens of stifle synovial membrane were also collected from 12 healthy dogs with normal stifles and 14 dogs with experimentally-induced CCL rupture of 19 weeks duration.

PCR Analysis for Bacterial DNA. Synovial fluid cells were centrifuged to isolate the cell pellet. DNA was extracted from synovium and from the synovial fluid cell pellet using standard TRIzol methods. A non-nested broad-range panbacterial PCR method was used for detection of DNA from the bacterial 16S rRNA gene. This PCR assay uses consensus primers for a highly conserved region of the gene. In addition, a species-specific non-nested PCR method was used to detect DNA from the Borrelia burgdorferi outer surface protein A gene (OspA). A species-specific nested PCR method was used to detect DNA from the Stenotrophomonas maltophilia 23S rRNA gene (23S rRNA). All PCR reactions were performed in a laminar flow hood and extensive precautions were taken to prevent contamination of the PCR assay. PCR products were examined in UV light after electrophoresis on a 1.5% agarose gel and staining with ethidium bromide.

Data Analysis. The Fisher's Exact test was used to compare the proportion of synovium and synovial fluid specimens that contained bacterial DNA for the panbacterial 16S rRNA and Stenotrophomoas maltophilia 23S rRNA genes, and to compare stifle joints of dogs with the CCL rupture arthropathy with normal and stifle instability control groups. The Kruskal-Wallis ANOVA test was used to determine whether variation in detection of bacterial DNA in the stifle joint occurred through year. Values of P<0.05 were considered significant.

Overall, when compared with controls (P<0.05), the presence of bacterial DNA within the stifle synovium was significantly associated with the CCL rupture arthropathy: 74% of joints were PCR-positive in dogs with the CCL rupture arthropathy, 36% of dogs with induced CCL rupture, and 11% of dogs with normal stifles and intact CCL.

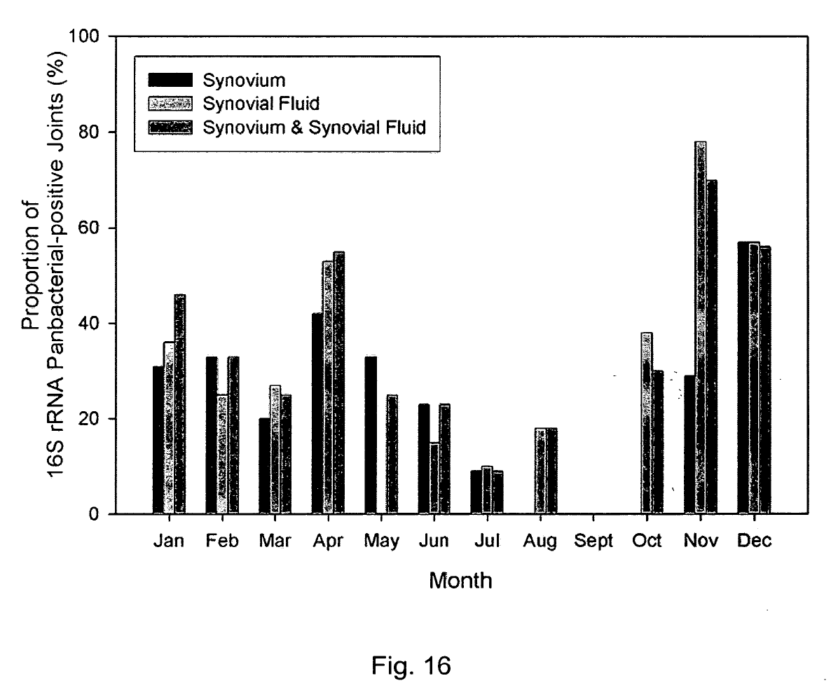

Detection of OspA DNA sequences was the least prevalent and detection of 23S rRNA Stenotrophomoas maltophilia DNA sequences was the most prevalent as shown in FIG. 16. Detection of OspA was not significantly different between groups, whereas detection of 16S rRNA sequences was associated with the CCL rupture arthropathy (P<0.05) when compared with control groups. Detection of 23S rRNA Stenotrophomonas maltophilia DNA sequences was also associated with the CCL rupture arthropathy when compared with dogs with normal stifles (P<0.05) but not dogs with induced CCL rupture (P=0.32).

Seasonal variation in the proportion of 16S rRNA PCR-positive joints (P=0.08 for synovium; P<0.05 for synovial fluid) and OspA PCR-positive joints (P<0.01 for synovium; P=0.07 for synovial fluid) was also found. Detection of 16S rRNA bacterial DNA sequences was most prevalent in the fall and winter and less prevalent in the late spring and summer, whereas detection of OspA DNA sequences was most prevalent in the spring and summer.

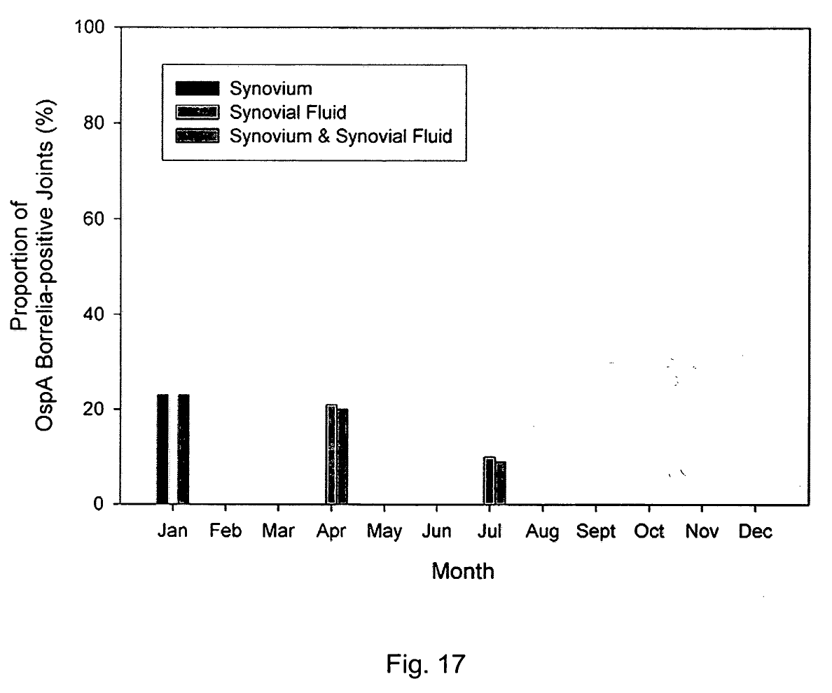

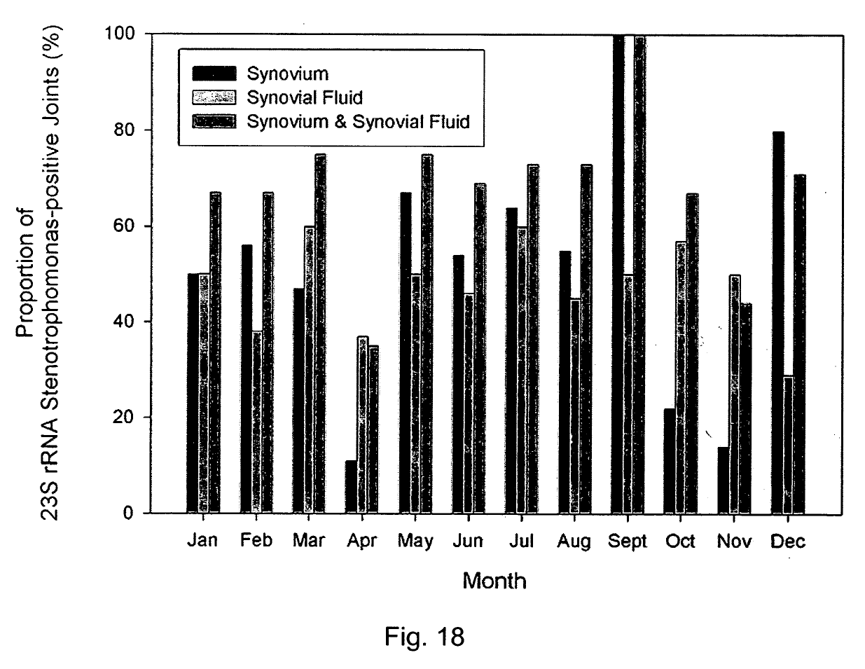

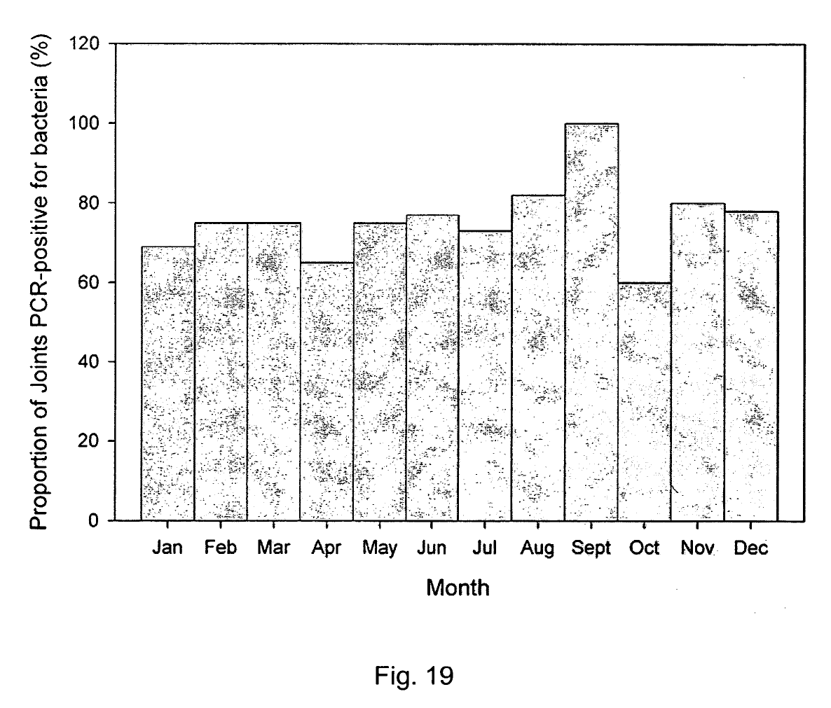

In contrast, variation in detection of 23S rRNA DNA sequences from Stenotrophomoas maltophilia was less evident (P<0.01 for synovium; P=0.98 for synovial fluid) throughout the year. There was a significant association between PCR detection of bacterial DNA in synovium and synovial fluid for the 16S rRNA gene (P<0.001), but not the 23S rRNA Stenotrophomonas maltophilia gene (P=0.056). In general, bacterial DNA sequences were detected more frequently in synovial fluid when compared with synovium (32% vs. 24% for panbacterial 16S rRNA; 4% vs. 2.6% Borrelia-OspA (FIG. 17); 47% vs. 47% Stenotrophomonas-23S rRNA (FIG. 18)).

DNA from a wide variety of bacterial species can often be found in the synovium of joints of both human and canine patients with aseptic forms of arthritis. Mixtures of bacterial DNA were detected in specimens of synovium from dogs with stifle arthritis and degenerative CCL rupture. The presence of DNA from Stenotrophomonas maltophilia within the stifle joint was also associated with the CCL rupture arthropathy when compared with control groups. Stenotrophomonas maltophilia is a ubiquitous environmental bacterium. It is hypothesized that mixtures of bacterial DNA are an important causative factor in the pathogenesis of the inflammatory stifle arthritis associated with the CCL rupture arthropathy. Chronic synovitis is known to degrade CCL structural properties over time.

Non-nested and nested PCR assays and analysis of both synovium and synovial fluid were conducted. It was found that bacterial DNA sequences were detectable in the stifle joints of the majority of affected dogs. Using a nested PCR assay for Stenotrophomonas maltophilia, it was found that Stenotrophomonas-specific 23S rRNA DNA sequences were particularly prevalent, which was expected since nested PCR doubles the number of PCR amplification cycles and increases both sensitivity and specificity of the PCR reaction. It was also found that using the nested assay, Stenotrophomonas DNA was also detectable in dogs with induced CCL rupture suggesting that stifle instability may promote translocation of bacterial material to the stifle joint over time.

It was also found that the prevalence of 16S rRNA and OspA DNA sequences in stifle joints varies according to the time of the year. Detection of OspA in the spring and early summer was expected given that Borrelia burgdorferi is vector-transmitted via Ixodes ticks. Interestingly, 16S rRNA DNA sequences also showed seasonal variation with the highest detection rates being in the late fall and winter months, which suggests that translocation of other types of bacterial material to joints may also involve an insect vector. Non-nested PCR data exhibited seasonal variation. Detection of 23S rRNA Stenotrophomonas maltophila DNA sequences using a nested PCR assay did not exhibit seasonal variation, which suggests that the amount of bacterial material within the stifle joint may be quite variable.

Use of real-time quantitative PCR assays could be used to estimate bacterial DNA sequence copy number within joints. Cloning and sequencing of the 16S rRNA PCR products may also determine how mixtures of bacterial material may vary throughout the calendar year. Borrelia burgdorferi is recognized as a joint pathogen of both humans and dogs. Borrelia burgdorferi is typically found together with mixtures of environmental bacteria in the canine stifle joint suggesting that mixtures of bacteria is important for development of synovitis. Although Stenotrophomonas maltophilia is considered an opportunistic pathogen of immunocompromized human beings, it is not currently recognized as a pathogen of the dog.

### Example 5

Specimens of peripheral blood and stifle synovial fluid were collected from 27 dogs with oligoarthritis and degenerative CCL rupture during surgical treatment. Mid-substance rupture was confirmed at the time of surgical treatment; none of the dogs had a history of traumatic injury. As a baseline control, synovial fluid specimens were collected from both stifles of 14 young healthy Beagles with intact CCL at the time of euthanasia, together with specimens of peripheral blood. Euthanasia was performed for reasons unrelated to the present study.

Synovial fluid samples from the left and right stifles of each dog were pooled for further analysis. Twelve of these dogs were female and two were male. The median age was 1 year (range 0.6-6) years, and body weight was 9.1±2.2 kg, mean+standard deviation. Finally, blood and synovial fluid specimens were also collected from 9 dogs without CCL rupture and with degenerative arthritis of a large synovial joint during surgical treatment.

Blood and Synovial Fluid Samples. Peripheral blood mononuclear cells (PBMC) were isolated using commercial cell separation tubes (BD Vacutainer™ CPT™, Becton Dickinson, Franklin Lakes, N.J.). Synovial fluid cells were isolated by centrifugation. Cells were stored at −80° C. for gene expression analysis.

Quantitative Reverse Transcriptase-Polymerase Chain Reaction (qRT-PCR). For RNA extraction, PBMC or synovial fluid cells were mixed with 1 ml of Trizol reagent (Invitrogen, Carlsbad, Calif.). After incubation at approximately 25° C. (room temperature, RT) for 5 minutes, 200 μl chloroform (Sigma Chemical Co, St Louis, Mo.) was added and the mixture was shaken vigorously by hand for 15 seconds and then incubated at RT for a further 10 minutes.

The aqueous phase was separated by centrifugation at 4° C. and 12,000 rpm for 15 minutes. The aqueous phase was then mixed with 500 μl of isopropyl alcohol (Fisher Scientific, Hannover Park, Ill.) in a new microtube. The mixture was incubated at RT for 10 min. and then centrifuged at 4° C. and 12,000 rpm for 10 minutes.

The pellet was washed with 1 ml of 75% isopropyl alcohol (Fisher Scientific, Hannover Park, Ill.) and centrifuged at 4° C., 8600 rpm for 5 minutes. The pellet was then air dried for 10 min. at RT and dissolved in 100 μl of RNase-free water. Total RNA was further purified using a RNA clean-up kit (Qiagen, Valencia, Calif.). cDNA was generated from 0.2-1 μg of total RNA by using the superscript III first-strand synthesis system for reverse transcriptase polymerase chain reaction (RT-PCR) (Invitrogen, Carlsbad, Calif.).

Quantitative real-time PCR was performed using standard methods and a MyiQ Real-Time PCR detection system (Bio-Rad, Hercules, Calif.). A panel of oligonucleotide primers was designed for the following genes: Cathepsin K, cathepsin S, matrix metalloproteinase-9 (MMP-9, gelatinase B), TRAP, invariant chain (li), Toll-like receptor-2 (TLR-2) and TLR-9 (See Table 1). Primers were designed from known canine gene sequences or regions of homology between the specific genes of other higher mammals. The 18S rRNA gene was used as the housekeeping gene.

All PCR reactions were carried out in a final volume of 25 μl, which contained 12.5 μl of 2× SYBR Green (Bio-Rad, Hercules, Calif.), 1 μl of 10 μM forward primer, 1 μl of 10 μM reverse primer, 1 μl of cDNA and 9.5 μl of DEPC water. PCR cycling conditions were 5 min. at 94° C. and 40 cycles of 94° C. for 30 sec., 60° C. for 30 sec. and 72° C. for 30 seconds.

Statistical Analysis. For each sample, the threshold cycle (Cₜ values) obtained from the exponential region of the PCR amplification plot from duplicate trials were averaged together. Relative gene expression for each of the genes-of-interest was then calculated using the −ΔΔCₜ method. (Livak K J et al., Analysis of relative gene expression data using real-time quantitative PCR and the 2-^{ΔΔCt} method, Methods 2001, 25:402-408).

PBMC gene expression was used as an internal control and the 18S rRNA gene was used as the housekeeping gene. Relative mRNA expression was calculated as 2^{-(average2ΔΔCt). Data were not normally distributed, therefore, the Kruskall-Wallis ANOVA test was used to determine differences between groups. After log-transformation, a Student's t test with a hypothesized mean equal to zero was used to determine whether synovial fluid gene expression was significantly different from PBMC (internal control). Differences were considered significant at P<}0.05.

Results. Signalment and clinical diagnosis. Breeds of dog affected with stifle oligoarthritis and degenerative CCL rupture were Labrador retriever (n=10), Labrador retriever mix (n=2), mastiff (n=2), Husky mix (n=2), Norwegian Elkhound (n=1), Chesapeake Bay Retriever (n=1), Anatolian Shepherd (n=1), Rottweiler (n=1); German Shorthair Pointer (n=1); German Shepherd dog (n=1); Weimeraner (n=1), Dobmerman (n=1), Springer Spaniel (n=1), Akita (n=1), and Pit Bull Terrier mix (n=1).

Body weight and age were 39.7±8.8 kg and 6±2 years, respectively. Fourteen dogs were ovariohysterectomized females, 12 dogs were castrated males, and 1 dog was male. Median duration of lameness was 12 weeks (range 1-168 weeks). Bilateral stifle arthritis and associated degenerative CCL rupture was found in 13 of 27 dogs (48%) clinically.

Breeds of dog affected with other forms of arthritis were Labrador retriever (n=3), Bernese Mountain dog (n=2), Labrador retriever cross (n=1), Boxer (n=1), Sharpei (n=1), and German Shepherd dog (n=1). Body weight and age were 30.4±10.6 kg and 1±0.6 years respectively. Two dogs were ovariohysterectomized females, 4 dogs were castrated males, 1 dog was male, and 2 dogs were female. Median duration of lameness was 12 weeks (range 6-26 weeks).

Synovial fluid was collected from the elbow joint of 6 dogs with arthritis associated with fragmented coronoid process, the hip joint of two dogs with arthritis associated with hip dysplasia, and 1 dog with stifle arthritis associated with medial patella luxation.

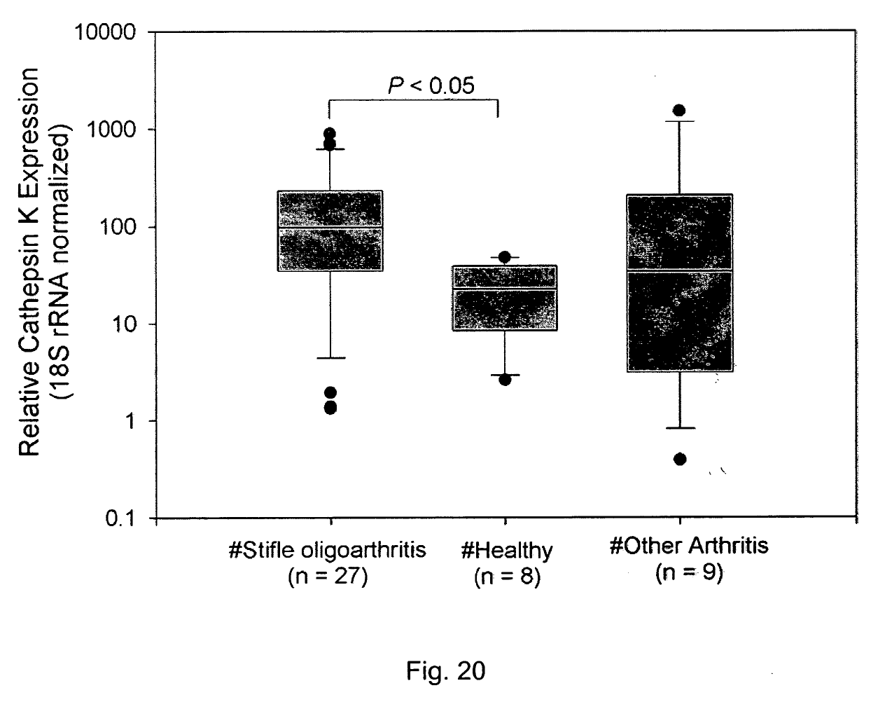

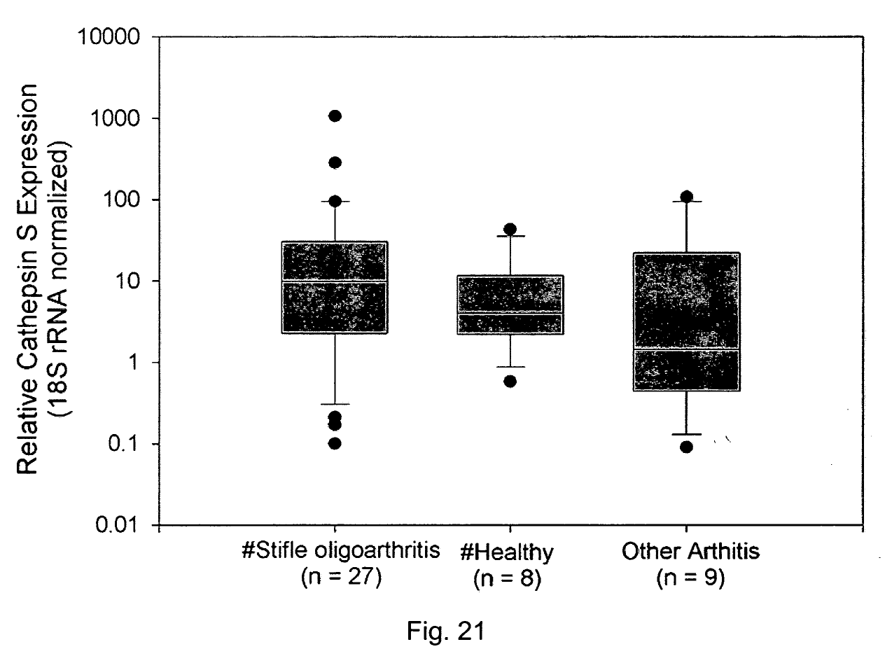

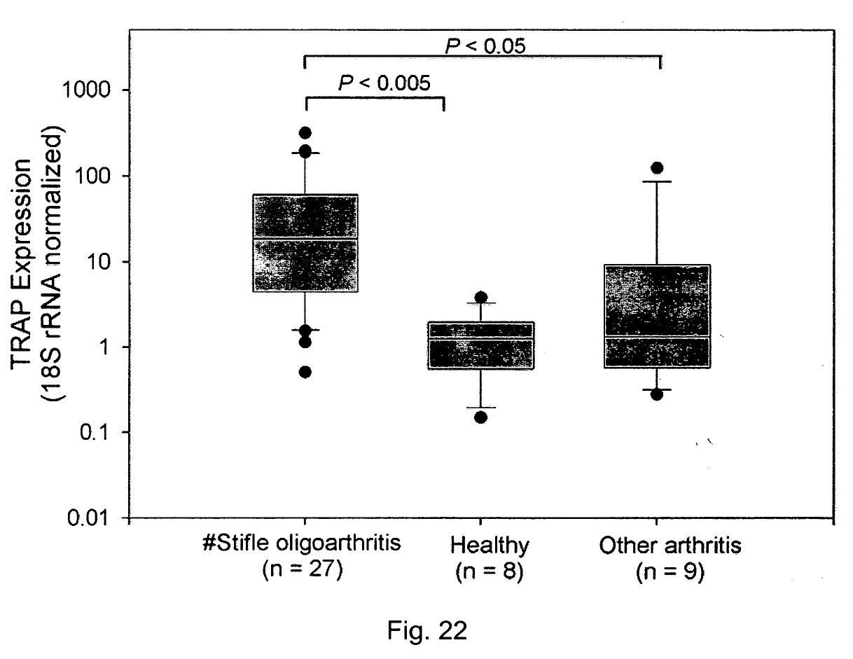

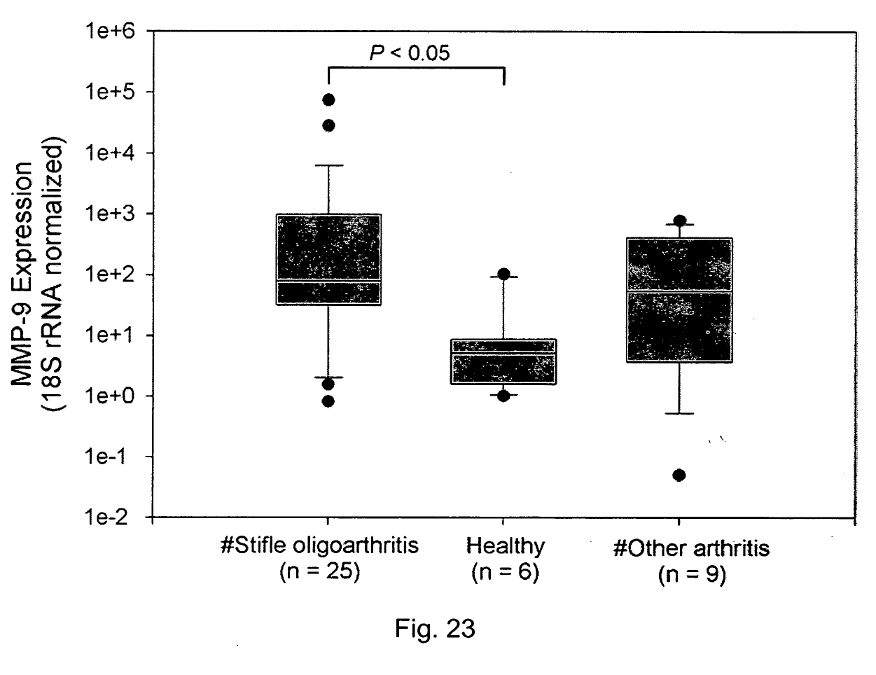

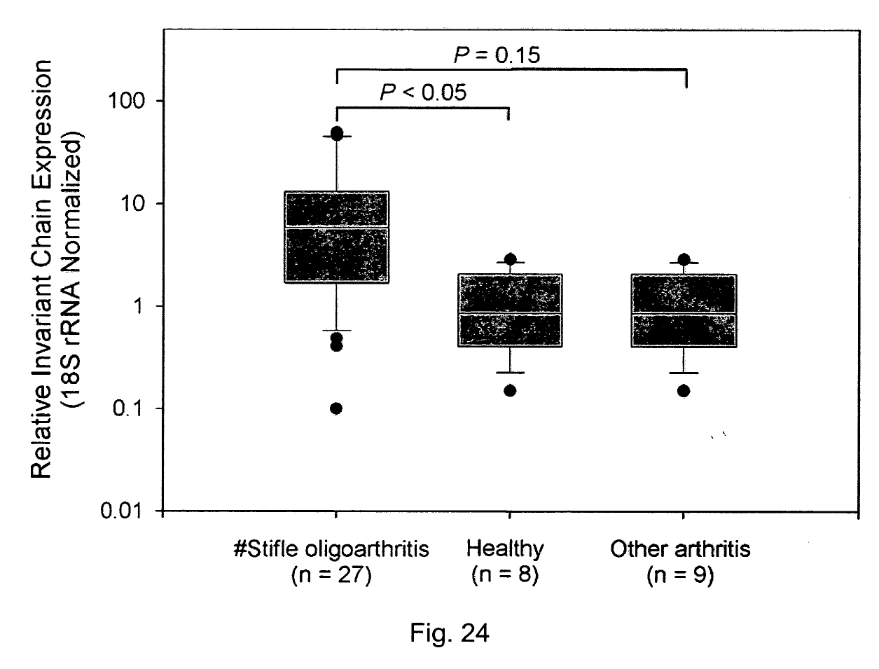

Relative gene expression. Relative gene expression results are summarized in FIGS. 20-26. Relative expression of cathepsin K (68-fold), MMP-9 (232-fold), TRAP (36-fold), and li (10-fold) genes was significantly increased in the stifle synovial fluid of dogs with oligoarthritis as compared with the stifles of healthy dogs (P<0.05). Relative expression of TLR-2 was also higher in synovial fluid of dogs with stifle oligoarthritis as compared with healthy dogs (P=0.08).

In contrast, relative expression of all of the genes-of-interest in synovial fluid from joints affected with other forms of arthritis was not significantly different from the stifle joints of healthy dogs. Expression of TRAP in the stifle joints of dogs with oligoarthritis was also increased as compared to joint expression of TRAP in dogs with other forms of arthritis (P<0.05).

In the dogs with stifle oligoarthritis, expression of cathepsin K (191-fold), cathepsin S (68-fold), MMP-9 (4771-fold), TRAP (53-fold), li (13-fold) and TLR-2 (40-fold) was significantly increased in stifle synovial fluid as compared with the internal PBMC control. In healthy dogs, only cathepsin K (24-fold), and cathepsin S (10-fold) expression was significantly increased in stifle synovial fluid as compared with the internal PBMC control (P<0.05). Expression of MMP-9 was also higher as compared with the internal PBMC control (P=0.05).

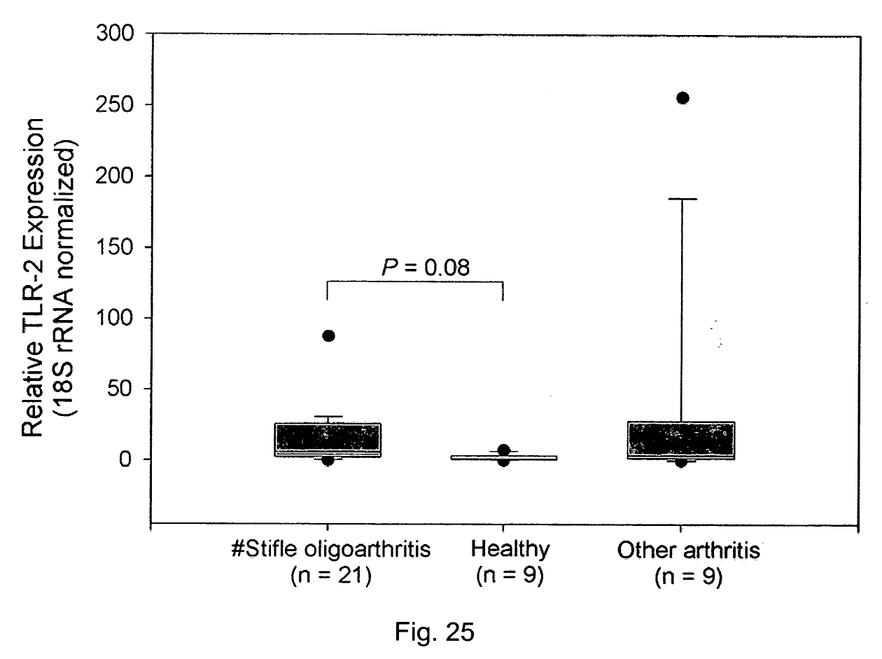

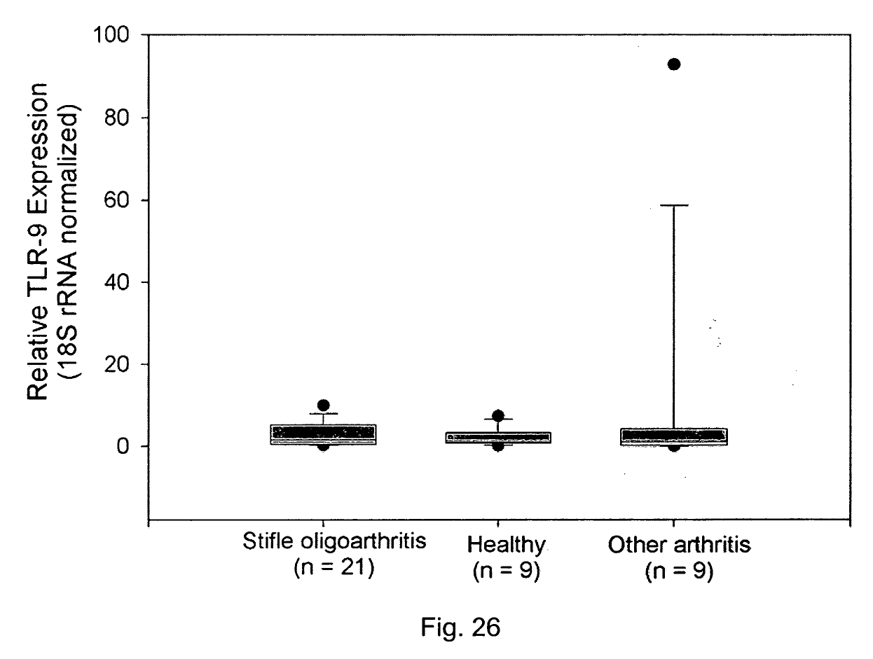

In dogs with other forms of arthritis, only expression of cathepsin K (257-fold) and MMP-9 (212-fold) was increased in synovial fluid as compared with the PBMC internal control (P<0.05). Expression of TLR-2 was also higher when compared with the internal PBMC control (P=0.1). In two different individual dogs with other forms of arthritis, high relative expression of TLR-2 and TLR-9 was found. (See FIGS. 25 and 26).

Although it has been recognized for some time that inflammation within the synovium of the stifle is typically found in dogs with CCL rupture, the importance of this pathological feature in the mechanism that leads to progressive rupture of the CCL and the development of severe stifle arthritis remains controversial.

Key features of the synovial inflammation include the presence of large numbers of dendritic cells, macrophages, and T lymphocytes (Lemburg A K et al., Immunohistochemical characterization of inflammatory cell populations and adhesion molecule expression in synovial membranes from dogs with spontaneous cranial cruciate ligament rupture, Vet Immunol Immunopathol 2004, 97:231-240); and, Muir P et al., Localization of cathepsin K and tartrate-resistant acid phosphatase in synovium and cranial cruciate ligament in dogs with cruciate disease, Vet Surg 2005, 34:239-246). Similar inflammatory changes are also found in adjacent CCL tissue. In this example, synovitis in dogs with stifle oligoarthritis and degenerative CCL rupture was associated with upregulation of TRAP and li, in particular.

In the group of young healthy dogs, expression of matrix turnover genes (cathepsin K, cathepsin S, and MMP-9) within the stifle joint was up-regulated compared with the PBMC internal control. This finding likely reflects normal turnover of matrix tissues within a large load-bearing synovial joint. In dogs with stifle oligoarthritis, expression of matrix turnover genes was also significantly increased as compared with internal PBMC control.

Over-expression of cathepsin K, cathespin S, and MMP-9 in joint tissues has been associated with development of joint degradation. (Hou W S et al., Comparison of cathepsins K and S expression within the rheumatoid and osteoarthritic synovium, Arthritis Rheum 2002, 46:663-674; Volk S W et al., Gelatinase activity in synovial fluid and synovium obtained from healthy and osteoarthritic joints of dogs, Am J Vet Res 2003, 64:1225-1233; Tsuboi H et al., Tartrate-resistant acid phosphatase (TRAP—positive cells in rheumatoid synovium may induce the destruction of articular cartilage, Ann Rheum Dis 2003, 62:196-203; and, Morko J et al., Spontaneous development of synovitis and cartilage degeneration in transgenic mice overexpressing cathepsin K, Arthritis Rheum 2005, 52:3713-3717).

In this example, MMP expression may be an important marker of joint degradation in dogs with oligoarthritis as joint expression relative to the PBMC internal control as more than 20-fold higher than in dogs with other forms of arthritis. Up-regulation of cathepsin K, cathepsin S, and MMP-9 within affected stifles may mediate progressive arthritic degradation of joint tissues and associated weakening eventual rupture of the CCL. Synovitis has been reported to induce progressive degradation of CCL structural properties over time. (Goldberg V M et al., The influence of an experimental immune synovitis on the failure mode and strength of the rabbit anterior cruciate ligament, J Bone and Joint Surg 1982, 64A:900-906).

Similarly, in large synovial joints from dogs with other forms of arthritis, cathepsin K and MMP-9 were also up-regulated, but cathepsin S was not. Here, up-regulation of cathepsin K and MMP-9 also likely mediates joint degradation.

Cathepsin S has a dual role in antigen presentation and matrix turnover in inflamed synovium (Hou et al., 2002). Cathepsin S has a critical role in degradation of li during antigen presentation in dendritic cells. (Nakagawa T Y et al., Impaired invariant chain degradation and antigen presentation and diminished collagen-induced arthritis in cathepsin S null mice, Immunity 1999, 10:207-217). Up-regulation of cathepsin S expression in stifle joints from dogs with oligoarthritis (as compared with the PBMC internal control) was found together with up-regulation of li and TRAP. Expression of TRAP and li within the stifle was also higher as compared with normal dogs.

Together, that suggests that antigen presentation by dendritic cells is increased within the stifle of affected dogs. Large numbers of MHC class II+ dendritic cells are typically found in the synovium of affected dogs. (Lemburg et al., 2004). TRAP is highly expressed in activated macrophages and dendritic cells and has a critical role in dendritic cell maturation and development of Th1 T lymphocyte responses to antigen. (Esfandiari E et al., TRACP influences Th1 pathways affecting dendritic cell function, J Bone Miner Res 2006, 21:1367-1379).

Th1 T lymphocytes may play an important role in the pathogenesis of stifle oligoarthritis because the Th1 cytokines interleukin-2 (IL-2) and interferon-γ are highly expressed, whereas little expression of the Th2 cytokine IL-4 is found. (Hegemann N et al., Cytokine profile in canine immune-mediated polyarthritis and osteoarthritis, Vet Comp Orthop Traumat 2005, 18:67-72). Because TRAP gene expression was specifically increased in dogs affected with stifle oligoarthritis (as compared with the other groups) quantification of TRAP type 5a protein may be useful as a biomarker of synovitis in this canine arthropathy. (Janckila A J et al., Properties and expression of human tartrate-resistant acid phosphatase isoform 5a by monocyte-derived cells, J Leukoc Biol 2005, 77:209-218). Identification of a clinically-relevant biomarker for stifle synovitis would facilitate identification of dogs at risk of developing CCL rupture for therapeutic intervention.

The triggering antigens involved in these antigen-specific immune responses in the stifles of dogs with oligoarthritis are unknown. Recently, bacterial material has been implicated as an immunologic trigger for synovitis in this condition. Development of immune responses to collagen type I neo-epitopes may also contribute to inflammatory responses within affected joints. (de Rooster H et al., Prevalence and relevance of antibodies to type-I and -II collagen in synovial fluid of dogs with cranial cruciate ligament rupture, Am J Vet Res 2003, 61:1456-1461; de Bruin T et al., Evaluation of anticollagen type I antibody titers in synovial fluid of both stifles and the left shoulder of dogs with unilateral cranial cruciate disease, Am J Vet Res 2007, 68:283-289; and, Muir P et al., Detection of DNA from a range of bacterial species in the stifle joints of dogs with inflammatory stifle arthritis and associated degenerative anterior cruciate ligament rupture, Micbob Pathog 2007, 42:47-55).

In this example, expression of TLR-2 is increased in the stifles of dogs with oligoarthritis (relative to the internal PBMC control) with higher expression (P=0.08) than in the stifles of healthy dogs, which suggests that TLR-2 signaling may be involved in the pathogenesis of synovitis in affected dogs. Some individual dogs with other forms of arthritis also had high levels of TLR expression within the affected joint.

TLR-2 is activated by bacterial lipoproteins and peptidoglycan. (Ishii M et al., Molecular cloning and tissue expression of canine toll-like receptor 2 (TLR2), Vet Immunol Immunopathol 2006, 110:87-95; Anders H-J et al., Molecular mechanisms of autoimmunity triggered by microbial infection, Arthritis Res Ther 2005, 7:215-224). A key role for TLR signaling is activation of dendritic cells to support Th1 T cell responses. (Anders et al., 2005). Such a role in canine oligoarthritis is also supported by the instant TRAP expression data and by the discovery that bacterial material is often found in affected stifles. (Muir et al., 2007).

Relatively simple quantitative RT-PCR methods were use to study gene expression in synovial tissue. Use of Taqman PCR conditions and standard curves could provide an additional level of specificity and may have reduced variance in gene expression data. (Boyle D L et al., Quantitative biomarker analysis of synovial fluid gene expression by real-time PCR, Arthritis Res Ther 2003, 5:R352-R360). Use of other PCR methods or refinements in primer design may also improve assay sensitivity.

The volume of synovial fluid that can be collected was often small, particularly from the joints of healthy dogs. This limited the number of genes that could be studied and all genes could not be assayed in the samples. The instant data are associative, but the instant findings do suggest that signaling of immune response genes may be involved in the pathogenesis of canine stifle oligoarthritis.

All of the dogs with oligoarthritis in the example had CCL-rupture confirmed during surgical treatment. Evaluation of stifle joint gene expression earlier in the course of rupture development (particularly in dogs with mechanically stable stifles) may provide further understanding of the pathogenesis of progressive CCL rupture.

Expansion of the panel of genes studied to include pro- and anti-inflammatory cytokines and assay of relevant protein products may also be important in future studies focused on biomarker determination. All tissue samples from baseline control dogs were from a younger population of Beagles. Whilst it is possible that breed- and age-specific differences in expression of the genes of interest may have influenced the results, use of Beagles as the baseline control is appropriate. The Beagle is a breed that is susceptible to oligoarthritis and associated degenerative CCL rupture. (Whitehair J G et al., Epidemiology of cranial cruciate ligament rupture in dogs, J Am Vet Med Assoc 1993, 203:1016-1019).

Surgical treatment for dogs with stifle instability secondary to CCL rupture is a most common surgical procedure performed in canine orthopedics. However, despite the use of various surgical procedures (including tibial plateau leveling osteotomy) limb function after surgery is relatively poor. (Conzemius M G et al., Effect of surgical technique on limb function after surgery for rupture of the cranial cruciate ligament in dogs, J Am Vet Med Assoc 2005, 226:232-236).

We claim:

1. A method of diagnosing persistent, chronic synovitis in the joint of a mammal comprising:
providing a test sample comprising synovial fluid, cell or tissue from the joint of a mammal,
quantifying one or more protein or mRNA biomarkers selected from the group consisting of cathepsin K, MMP-2, MMP-9, cathepsin S, tartrate-resistant acid phosphatase, invariant chain, CD4⁺ T-lymphocytes, CD8⁺ T-lymphocytes, CD44⁺ mononuclear cells, Toll-like receptor-2, Toll-like receptor-9, and combinations thereof, and,
comparing the amount of the one or more biomarkers from the test sample to a corresponding biomarker concentration in an internal control sample,
wherein a statistically significant elevated concentration of the one or more biomarkers in the test sample indicates that the mammal's joint is diseased with persistent, chronic synovitis.

2. A method of diagnosing progressive joint degradation in the joint of a mammal comprising:
providing a test sample comprising synovial fluid, cell or tissue from the joint of a mammal,
quantifying the amount of one or more protein or mRNA biomarkers selected from the group consisting of cathepsin K, MMP-2, MMP-9, cathepsin S, tartrate-resistant acid phosphatase, invariant chain, CD4⁺ T-lymphocytes CD8⁺, T-lymphocytes, CD44⁺ mononuclear cells, Toll-like receptor-2, Toll-like receptor-9, and combinations thereof, and,
comparing the amount of the one or more biomarkers from the test sample to a corresponding biomarker concentration in an internal control sample,
wherein a statistically significant elevated concentration of the one or more biomarkers in the test sample indicates that the mammal's joint is diseased with progressive joint degradation.

3. The method of claims 1 or 2, wherein the internal control sample comprises peripheral blood mononuclear cells.

4. The method of claims 1 or 2, wherein the elevated concentration is statistically significant at P<0.05.

5. The method of claims 1 or 2, wherein the mammal is a dog.

6. The method of claims 1 or 2, wherein the mammal is a human.

7. The method of claims 1 or 2, wherein the synovial fluid, cell or tissue contains bacterial DNA generated by one or more members selected from the group consisting of Borrelia burgdorferi, Stenotrophomonas maltophilia, uncultured Eubacterium, Rhizobium radiobacter, Ralstonia solanacearum, uncultured beta Proteobacterium, Achromobacter xylosoxidans, uncultured Oxalobacterium, Corynebacterium glutamicum, Rhizobium galegae, Gordonia terrae, Acinetobacter calcoaceticus, Pseudomonas putida, uncultured Gemmatiomonadates bacterium, and, uncultured Burkholderia.

8. The method of claim 7, wherein the bacterium is Borrelia burgdorferi.

9. The method of claim 7, wherein the bacterium is Stenotrophomonas maltophilia.

10. The method of claim 5, wherein the dog has the major histocompatibility complex class II alleles DLA-DRB1*0102 and DLA-DRB1*1502.

11. The method of claims 1 or 2, wherein one or more biomarkers are in the form of mRNA.

12. The method of claim 1 or 2, wherein the biomarker is a member selected from the group consisting of cathepsin K, MMP-2 and -9, cathepsin S, tartrate-resistant acid phosphatase, invariant chain and combinations thereof, and wherein the concentration of the one or more biomarkers are measured using RNAzol B methodology including quantitative RT-PCR.

13. The method of claim 1 or 2, wherein the concentration of cathepsin K is elevated around 167-fold compared with the internal peripheral blood mononuclear cells control.

14. The method of claim 1 or 2, wherein the concentration of MMP-9 is elevated around 4053-fold, compared with the internal peripheral blood mononuclear cells control.

15. The method of claim 1 or 2, wherein the concentration of cathepsin S is elevated around 60-fold, compared with the internal peripheral blood mononuclear cells control.

16. The method of claim 1 or 2, wherein the concentration of tartrate-resistant acid phosphatase is elevated around 51-fold, compared with the internal peripheral blood mononuclear cells control.

17. The method of claim 1 or 2, wherein the concentration of invariant chain is elevated around 15-fold, compared with the internal peripheral blood mononuclear cells control.

18. The method of claim 1 or 2, wherein the biomarker is a member selected from the group consisting of bacterial peptidoglycan, CD4⁺ T-lymphocytes, CD8⁺ T-lymphocytes, CD44⁺ mononuclear cells, Toll-like receptor-2, Toll-like receptor-9, and combinations thereof, and wherein the concentration of the one or more biomarkers are measured using flow cytometry.

19. The method of claim 1 or 2, wherein the biomarker is a member selected from the group consisting of peptidoglycan⁺ mononuclear cells, CD4⁺ T-lymphocytes, CD8⁺ T-lymphocytes, CD44⁺ mononuclear cells, Toll-like receptor-2, Toll-like receptor-9, and combinations thereof, and wherein the concentration of the one or more biomarkers are measured using an enzyme-linked immunosorbent assay.

20. A method of diagnosing synovitis in the joint of a mammal comprising:
providing a test sample comprising synovial fluid, cell or tissue from the joint of a mammal, and, detecting the presence of bacterial DNA or mixtures of bacterial DNA.

21. The method of claim 20, wherein the mammal is a dog.

22. The method of claim 20, wherein the mammal is a human.

23. The method of claim 20, wherein bacterial DNA is generated by one or more members selected from the group consisting of Borrelia burgdorferi, Stenotrophomonas maltophilia, uncultured Eubacterium, Rhizobium radiobacter, Ralstonia solanacearum, uncultured beta Proteobacterium, Achromobacter xylosoxidans, uncultured Oxalobacterium, Corynebacterium glutamicum, Rhizobium galegae, Gordonia terrae, Acinetobacter calcoaceticus, Pseudomonas putida, uncultured Gemmatiomonadates bacterium, and, uncultured Burkholderia.

24. A method of diagnosing genetic predisposition to inflammatory arthritis in a host dog comprising:
providing a test sample containing host dog DNA, and,
determining the HLA-DRB1 or DLA-DRB1 allele.

25. The method of claim 24, wherein the host dog has major histocompatibility complex class II alleles.

26. The method of claim 25, wherein the major histocompatibility complex class II alleles are a member selected from the group consisting of DLA-DRB1*0102 and DLA-DRB1*1502.

27. A method of treating 16S rRNA PCR-positive joints in a mammal comprising administering to the mammal a therapeutic amount of a tetracycline.

28. The method of claim 27, wherein the tetracycline is doxycycline.
